(19) United States
(12) Patent Application Publication
(10) Pub. No.: US 2010/0273267 A1
(43) Pub. Date: Oct. 28, 2010

(54) DIAGNOSIS OF ABDOMINAL AORTIC ANEURYSM USING BIOMARKERS

(76) Inventor: Gregory S. Hageman, Salt Lake City, UT (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/681,611

(22) PCT Filed: Oct. 7, 2008
(86) PCT No.: PCT/US08/79105

§ 371 Date: Apr. 2, 2010

Related U.S. Application Data
(60) Provisional application No. 60/978,326, filed on Oct. 8, 2007.

(51) Int. Cl. G01N 33/68

(52) U.S. Cl. 436/86

(57) ABSTRACT

The invention relates to proteins associated with abdominal aortic aneurysm (AAA). These proteins, which are present in blood, are expressed in individuals with AAA at either elevated or reduced levels compared to healthy individuals. The invention provides methods for diagnosing AAA. The invention provides methods for determining the efficacy of preventive treatment for AAA. The invention provides methods for monitoring the progression of AAA

### CROSS-REFERENCE TO RELATED APPLICATIONS

This application claims priority to U.S. provisional application No. 60/978,326 filed Oct. 8, 2007, the entire content of which is incorporated herein by reference.

### STATEMENT AS TO RIGHTS TO INVENTIONS MADE UNDER FEDERALLY SPONSORED RESEARCH AND DEVELOPMENT

This invention was made with government support under NIH grant R01 EY11515. The government has certain rights in the invention.

### FIELD OF THE INVENTION

The invention relates to diagnosis of abdominal aortic aneurism and has application in biology and medicine.

### BACKGROUND OF THE INVENTION

Pathological changes associated with many disorders or conditions are reflected in the protein profile of serum and plasma (because blood comes into contact with most of the tissues in the human body), as well as other body fluid, such as urine. Monitoring the levels (and changes in levels) of such proteins, or “biomarkers” is useful for diagnosis and prognosis of diseases, disorders or conditions. In addition, changes in levels of biomarker can serve as surrogate endpoints for assessing the effects and efficacy of therapeutic interventions.

An aortic aneurysm is a vascular disorder involving swelling or stretching of the aorta resulting from weakness in the aortic wall. Although stretching of the aorta may cause physical discomfort, the serious medical risk is rupture of the aorta, which causes severe pain, internal bleeding and, absent prompt treatment, death. Aneuryms are also a source of blood clots, which can cause many complications, including a heart attack or stroke. The most common aneurysm is abdominal aortic aneurysm (AAA), which occurs in the abdominal aorta that supplies blood to the abdomen, pelvis and legs.

AAA develops slowly over time and is most common in older individuals, with the average age at diagnosis being 65-70 years. Risk factors for AAA include high blood pressure, smoking, cholesterol and obesity. AAA is currently diagnosed by abdominal ultrasound, abdominal CT scanning and aortic angiography. Therapeutic options are available for individuals with AAA, including surgical replacement of the abdominal vessel and endovascular stent grafting, and others are being developed. There is a need, however, for additional treatments. Similarly, there is a need for new methods for diagnosis and progression of AAA. The present invention addresses these and other needs.

### BRIEF SUMMARY OF THE INVENTION

The invention relates to proteins associated with abdominal aortic aneurysm (AAA). These AAA-associated proteins (biomarkers) are present in the serum or other blood fraction, urine, or other body fluid of individuals with AAA at elevated or reduced levels compared to healthy individuals (i.e., age-matched controls). The invention provides methods and kits for using the biomarkers for diagnosing AAA, assessing the efficacy of preventive treatment for AAA or monitoring the progression of AAA, in an individual. As used herein, diagnosing AAA includes detecting the very early stages of AAA, even prior to the individual showing any symptoms or signs of the disorder.

The invention provides methods for diagnosing AAA, assessing the efficacy of preventive treatment for AAA or monitoring the progression of AAA by determining the levels of biomarkers in a biological sample from an individual and comparing the levels of the biomarkers to earlier determined levels or reference levels of the biomarkers. Determination that a biomarker is at a level characteristic of a vascular disorder in a subject suggests that the tested subject has or may be developing the disorder (i.e., AAA), while determination that a biomarker is at a level characteristic of a non-vascular disorder state in a subject suggests that the tested subject does not have or is not developing the disorder. Likewise, a change of biomarker levels over time to levels closer to that of a vascular disorder state suggests progression of the disorder (i.e., AAA), while change of biomarker levels over time to levels closer to that of a non-vascular disorder state suggests regression of the disorder (e.g., therapeutic efficacy).

In one embodiment, the methods for diagnosing AAA, assessing the efficacy of preventive treatment for AAA or monitoring the progression of AAA involve determining the levels of biomarkers in a biological sample from an individual and comparing the level of the biomarkers to earlier determined levels and/or to reference levels of the biomarkers

In one embodiment, the biological sample is from the serum of an individual. In another embodiment, the biological sample is from the plasma of an individual. In another embodiment, the biological sample is from the urine of an individual. In one embodiment, the biological sample is depleted of albumin and IgG.

As used herein, the biomarkers for diagnosing AAA, assessing the efficacy of preventive treatment for AAA or monitoring the progression of AAA in an individual are proteins, which may be identified and characterized by their mass-to-charge ratio as determined by mass spectrometry, as indicated in Tables 1-4.

The biomarkers of the invention are listed in Tables 1-4.

In certain embodiments, the levels of a combination of biomarkers (i.e., a set of biomarkers) as described herein are determined, e.g., the levels of 1, at least 2, at least 3, at least 4, at least 5, at least 6, at least 7, at least 8, at least 9, or at least 10 of the biomarkers listed in Tables 1-4.

In certain embodiments, the levels of a set of biomarkers as described herein are determined. The sets of biomarkers have shared properties, e.g., presence at elevated levels in individuals diagnosed with AAA compared to controls, presence at reduced levels in individuals diagnosed with AAA compared to controls, ratio or difference in levels between individuals diagnosed with AAA and controls (e.g., between 1.25- and 2-fold, between 2- and 3-fold, between 3- and 5-fold, or at least 5-fold difference between levels in individuals diagnosed with AAA compared to controls), source of the biological sample containing the biomarkers, method used to identify and characterize the biomarkers, function, or any combination of these properties.

In one embodiment, the biomarkers in a set of biomarkers are selected from the biomarkers present at different levels in the serum of individuals diagnosed with AAA as compared to a control population, e.g., biomarkers listed in Table 1.

In one embodiment, the biomarkers in a set of biomarkers are selected from the biomarkers present at different levels in albumin and IgG deleted serum of individuals diagnosed with AAA as compared to a control population, e.g., biomarkers listed in Table 2.

In one embodiment, the biomarkers in a set of biomarkers are selected from the biomarkers present at different levels in albumin and IgG deleted plasma of individuals diagnosed with AAA as compared to a control population, e.g., biomarkers listed in Table 3.

In one embodiment, the biomarkers in a set of biomarkers are selected from the biomarkers present at different levels in the urine of individuals diagnosed with AAA as compared to a control population, e.g., biomarkers listed in Table 4.

In one embodiment, the biomarkers in a set of biomarkers are selected from the biomarkers present at elevated levels in individuals diagnosed with AAA as compared to a control population, e.g., biomarkers listed as “i” in Tables 1-4.

In one embodiment, the biomarkers in a set of biomarkers are selected from the biomarkers present at reduced levels in individuals diagnosed with AAA as compared to a control population, e.g., biomarkers listed as “i” in Tables 1-4.

In one embodiment, at least one biomarker is a biomarker present in serum at significantly different levels in individuals with or developing AAA, compared to age-matched controls, selected from the following biomarkers in Table 1: 2685; 3350; 4708; 11573; 11643; 14564; 11687; 12545; 14608; and 53715.

In one embodiment, at least one biomarker is a biomarker present in serum at significantly elevated levels in individuals with or developing AAA, compared to age-matched controls, selected from the following biomarkers in Table 1: 2685; 11573; 11643; 14564; 11687; 14608; and 53715.

In one embodiment, at least one biomarker is a biomarker present in serum at significantly reduced levels in individuals with or developing AAA, compared to age-matched controls, selected from the following biomarkers in Table 1: 3350; 4708; and 12545.

In one embodiment, at least one biomarker is a biomarker present in albumin and IgG depleted serum at significantly different levels in individuals with or developing AAA, compared to age-matched controls, selected from the following biomarkers in Table 2: 3195; 3504; 3642; 3881; 108804; 15480; 3003; 3061; 3233; 3685; 4490; 4603; 7698; 9211; and 39702.

In one embodiment, at least one biomarker is a biomarker present in albumin and IgG depleted serum at significantly elevated levels in individuals with or developing AAA, compared to age-matched controls, selected from the following biomarkers in Table 2: 3195; 3003; 3061; 3685; 4490; and 39702.

In one embodiment, at least one biomarker is a biomarker present in albumin and IgG depleted serum at significantly reduced levels in individuals with or developing AAA, compared to age-matched controls, selected from the following biomarkers in Table 2: 3504; 3642; 3881; 108804; 15480; 3233; 4603; 7698; and 9211.

In one embodiment, at least one biomarker is a biomarker present in albumin and IgG depleted plasma at significantly different levels in individuals with or developing AAA, compared to age-matched controls, selected from the following biomarkers in Table 3: 3247; 3285; 3443; 3508; 4030; 4632; 4750; 11635; 14579; 66262; 3092; 3284; 3303; 3458; 3681; 3867; 3943; 4493; 4602; 6072; 6412; 6559; 6608; 10791; 11631; 11677; and 14626.

In one embodiment, at least one biomarker is a biomarker present in albumin and IgG depleted plasma at significantly elevated levels in individuals with or developing AAA, compared to age-matched controls, selected from the following biomarkers in Table 3: 4030; 11635; 14579; 66262; 3092; 3681; 3867; 4493; 6072; 6412; 6559; 6608; 10791; 11631; 11677; and 14626.

In one embodiment, at least one biomarker is a biomarker present in albumin and IgG depleted plasma at significantly reduced levels in individuals with or developing AAA, compared to age-matched controls, selected from the following biomarkers in Table 3: 3247; 3285; 3443; 3508; 4632; 4750; 3284; 3303; 3458; 3943; and 4602.

In one embodiment, at least one biomarker is a biomarker present in urine at significantly different levels in individuals with or developing AAA, compared to age-matched controls, selected from the following biomarkers in Table 4: 3742; 3856; 4458; 5716; 2746; 220; and 423.

In one embodiment, at least one biomarker is a biomarker present in urine at significantly elevated levels in individuals with or developing AAA, compared to age-matched controls, selected from the following biomarkers in Table 4: 3742; 3856; 5716; 2746; and 220.

In one embodiment, at least one biomarker is a biomarker present in urine at significantly reduced levels in individuals with or developing AAA, compared to age-matched controls, selected from the following biomarkers in Table 4: 4458 and 423.

In one embodiment, the biomarkers are measured by capturing the biomarker on an adsorbant of a surface enhanced laser desorption ionization (SELDI) probe and detecting the captured biomarkers by laser desorption-ionizing mass spectrometry. In one embodiment, the adsorbant is a cation exchange adsorbant, an anion exchange adsorbant, an immobilized metal affinity capture adsorbant, or a hydrophobic adsorbant. In one embodiment, the adsorbant is a biospecific adsorbant. In one embodiment, the biomarkers are measured using an immunoassay.

In one aspect, the invention provides a method for diagnosing AAA in an individual, by determining the levels of biomarkers in a biological sample from the individual, and comparing the levels of the biomarkers in the biological sample from the individual to reference levels of the biomarkers characteristic of a control population, where a difference in the levels of the biomarkers between the biological sample from the individual and the control population indicates that the individual is developing or has AAA. The methods may include the steps of obtaining a biological sample from the individual and determining the levels of the biomarkers. The methods may include determining the levels of a combination or set of biomarkers, for example, as described in paragraphs [0021] to [0032] above. The levels of certain biomarkers are significantly different in individuals with AAA than in healthy individuals. The levels of certain biomarkers are higher in individuals with AAA than in healthy individuals. The levels of certain biomarkers are lower in individuals with AAA than in healthy individuals.

In one embodiment, a method for diagnosing AAA in an individual involves obtaining a biological sample from the individual and determining the levels of the biomarkers by separating and detecting proteins by surface enhanced laser desorption ionization (SELDI).

The biomarkers can be obtained in a biological sample, preferably a fluid sample, of the individual. The biological sample can also be a tissue sample, e.g., a skin biopsy. The precise biological sample to be taken from an individual may vary, but the sampling is typically minimally invasive and is easily performed by conventional techniques known in the art. The at least two biomarkers are preferentially obtained in a biological sample of the individual's blood, serum, plasma, urine, cerebral spinal fluid (CSF), or saliva. The biological sample can be depleted of albumin and IgG, if appropriate.

In another aspect, the invention provides a method for assessing the efficacy of a preventive treatment for AAA in an individual, by determining the levels of biomarkers in a biological sample from the individual before treatment or at a first time point after treatment, determining the levels of biomarkers in the biological sample from the individual at a later time point during treatment or after treatment, and comparing the levels of the biomarkers at the two time points, where a difference in the levels of the biomarkers between the two determinations in which the levels of the biomarkers move closer to reference levels of the biomarkers characteristic of a control population indicates that the treatment is effective. The methods may include the steps of obtaining a biological sample from the individual and determining the levels of biomarkers as above. The methods may include determining the levels of a combination or set of biomarkers, for example, as described in paragraphs [0021] to [0032] above.

In one embodiment, a method for assessing the efficacy of preventive treatment for AAA in an individual involves the individual being treated with an agent effective to prevent or delay the disorder.

In one embodiment, a method for assessing the assessing the efficacy of preventive treatment for AAA in an individual involves obtaining a biological sample from the individual and determining the levels of the at least two biomarkers by separating and detecting proteins by SELDI.

In one embodiment, a method for assessing the efficacy of preventive treatment for AAA in a individual involves obtaining a biological sample of blood, serum, plasma or urine from the individual and determining the level of the at least two biomarkers.

In another aspect, the invention provides a method for monitoring the progression of AAA in an individual, comprising determining the levels of biomarkers in a biological sample from the individual, and comparing the levels of the biomarkers in the biological sample from the individual to reference levels of the biomarkers characteristic of a control population. In a related aspect, the invention provides a method for monitoring the progression of AAA in an individual, comprising determining the levels of biomarkers in a biological sample from the individual before treatment or at a first time point after treatment, determining the levels of biomarkers in the biological sample from the individual at a later time point during treatment or after treatment, and comparing the levels of the biomarkers at the two time points. In one embodiment, the individual is being administered with an agent effective to treat or prevent AAA, and the levels of the biomarkers determine the future treatment regime for the individual. The methods may include the steps of obtaining a biological sample from the individual and determining the levels of the biomarkers as above. The methods may include determining the levels of a combination or set of biomarkers, for example, as described in paragraphs [0021] to [0032] above.

In another aspect, the invention provides a kit comprising a solid support comprising at least one capture reagent attached thereto, wherein the capture reagent binds at least two biomarkers selected from the biomarkers indicated in Tables 1-4, at least two biomarkers selected from the biomarkers indicated in Tables 1-4, and instructions for using the solid support to detect the biomarkers contained in the kit.

In one embodiment, the solid support comprising the capture reagent is a SELDI probe. In one embodiment, the adsorbant is a cation exchange adsorbant, an anion exchange adsorbant, an immobilized metal affinity capture adsorbant, or a hydrophobic adsorbant. In one embodiment, the adsorbant is a biospecific adsorbant.

In one embodiment, the kit provides at least two biomarkers selected from the biomarkers indicated in Tables 1-4. In various embodiments, the kit contains a combination or set of biomarkers, for example, as described in paragraphs [0021] to [0032] above.

In one embodiment, the kit provides instructions for using the solid support to detect the biomarkers contained in the kit.

### BRIEF DESCRIPTION OF THE DRAWINGS

FIG. 1 shows a representative mass spectra displaying proteins present in albumin and IgG depleted plasma from 7 individuals diagnosed with AAA (top panel) and 7 age-matched controls (bottom panel). The potential biomarkers are detected using a CM10 pH4 array, a 50% SPA matrix, and high laser for data acquisition, according to the methods described in Example 6. The figure shows the mass-to-charge ratio (X-axis) and relative peak intensity (Y-axis) for a portion of the spectra.

### DETAILED DESCRIPTION OF THE INVENTION

The invention relates to biomarkers associated with abdominal aortic aneurysm (AAA). The biomarkers are proteins present in the serum or other blood fraction, urine, or other body fluid of individuals with AAA at elevated or reduced levels compared to healthy individuals (i.e., age-matched controls). Certain of these biomarkers are present at elevated levels in individuals with AAA compared to controls. Certain other of these biomarkers are present at reduced levels in individuals with AAA compared to controls.

In one aspect, the invention provides methods for diagnosing AAA by determining the levels of at least two biomarkers in an individual and comparing the levels of the at least two biomarkers with reference levels characteristic of a control, healthy population. In these methods, the levels of a combination or set of biomarkers, for example, as described in paragraphs [0021] to [0032], are determined. In one aspect, the invention provides methods for assessing the efficacy of preventive treatment for AAA by determining the levels of at least two biomarkers in an individual with AAA being treated for the disorder and comparing the levels of the at least two biomarkers to an earlier determined levels or reference levels of the biomarker. In these methods, the levels of a combination or set of biomarkers, for example, as described in paragraphs [0021] to [0032], are determined. In one aspect, the invention provides methods for monitoring the progression of AAA by determining the levels of at least two biomarkers in an individual with AAA and comparing the levels of the at least two biomarkers with reference levels characteristic of a control, healthy population. In a related aspect, the invention provides methods for monitoring the progression of AAA by determining the levels of at least two biomarkers in an individual with AAA being treated for the disease and comparing the levels of the at least two biomarkers to earlier determined levels or reference levels of the biomarker. In these methods, the levels of a combination or set of biomarkers, for example, as described in paragraphs [0021] to [0032], are determined.

### I. Definitions

The following definitions are provided to aid in understanding the invention. Unless otherwise defined, all terms of art, notations and other scientific or medical terms or terminology used herein are intended to have the meanings commonly understood by those of skill in the arts of medicine and molecular biology. In some cases, terms with commonly understood meanings are defined herein for clarity and/or for ready reference, and the inclusion of such definitions herein should not be assumed to represent a substantial difference over what is generally understood in the art.

“Biomarkers” are proteins present at different, i.e., reduced or elevated, levels in a biological fluid or tissue sample from individuals diagnosed with AAA compared to an age-matched control individual.

“Biological sample” refers to a fluid or tissue sample obtained from an individual that contains the biomarkers of the invention. The biological fluid sample can be, for example, a sample of an individual's blood, serum, plasma, urine, CSF or saliva. The biological tissue sample can be, for example, a skin biopsy. The biological sample can also be depleted of particular proteins, for example, albumin and IgG, if appropriate.

“Level” refers to the amount of a biomarker in a biological sample obtained from an individual. The level(s) of a biomarker(s) can be determined for a single biomarker or for a “set” of biomarkers. A set of biomarkers refers to a group of more than one biomarkers that have been grouped together, for example and not for limitation, by a shared property such as their presence at elevated levels in individuals diagnosed with AAA compared to controls, by their presence at reduced levels in individuals diagnosed with AAA compared to controls, by their ratio or difference in levels between individuals diagnosed with AAA and controls (e.g., between 1.25- and 2-fold, between 2- and 3-fold, between 3- and 5-fold, or at least 5-fold difference between levels in individuals diagnosed with AAA compared to controls), by the source of the sample containing the biomarkers, by the method used to identify and characterize the biomarkers, by function, or by any combination of these properties.

The level of the biomarker can be determined by any method known in the art and will depend in part on the nature of the biomarker. Methods for determining the level of a biomarker include surface enhanced laser desorption ionization (SELDI) mass spectrometry, electrophoresis (including capillary electrophoresis, 1- and 2-dimensional electrophoresis, 2-dimensional difference gel electrophoresis DIGE followed by MALDI-ToF mass spectrometry), chromatographic methods (such as high performance liquid chromatography (HPLC), thin layer chromatography (TLC), and hyperdiffusion chromatography), mass spectrometry (MS), various immunological methods (such as fluid or gel precipitin reactions, single or double immunodiffusion, immunoelectrophoresis, radioimmunoassay (RIA), enzyme-linked immunosorbant assays (ELISA), immunofluorescent assays, and Western blotting), and assays for an activity of a biomarker. It is understood that the level of the biomarker need not be determined in absolute terms, but can be determined in relative terms. For example, the level of the biomarker may be expressed as its concentration in a biological sample, as the concentration of an antibody that binds to the biomarker, or as the functional activity (i.e., binding or enzymatic activity) of the biomarker.

“Difference” as it relates to the level of a biomarker of the invention refers to a difference that is statistically different. A difference is statistically different, for example and not for limitation, if the expectation is <0.05, i.e., the p value determined using the Student's t-test is <0.05. The difference in level of a biomarker between an individual diagnosed with AAA and a control individual or population can be, for example and not for limitation, at least 10% different (1.10 fold), at least 25% different (1.25-fold), at least 50% different (1.5-fold), at least 100% different (2-fold), at least 200% different (3-fold), at least 400% different (5-fold), at least 10-fold different, at least 20-fold different, at least 50-fold different, at least 100-fold different, at least 150-fold different, or at least 200-fold different.

“Reference” as it relates to a biomarker of the invention refers to a level or amount of a biomarker in a healthy individual or control population. The reference level or amount may be determined by obtaining a biological sample and detecting the biomarker in a healthy individual, or may be determined by taking the level or amount known or readily determined from a control population.

“Control” refers to an individual who has not been diagnosed as having AAA and who has not displayed upon examination any symptoms characteristic of AAA, or a group of such individuals. An exemplary control population are age-matched individuals who have not been diagnosed as having AAA.

“Treating” or “treatment” refers to the treatment of a disorder or condition in a mammal, preferably a human, in which the disorder or condition has been diagnosed as AAA involving stretching of the aortic wall. Treating or treatment includes inhibiting the disorder or condition (i.e., arresting progression), relieving or ameliorating the disorder or condition (i.e., causing regression), or preventing or delaying progression of the disorder or condition. Treating or treatment can involve a course of treatment in which an individual with AAA is administered an agent more than once periodically over time that is expected to be effective in inhibiting, relieving or ameliorating, preventing or delaying progression of the disorder.

“Agent” refers to a drug or drug candidate. An agent may be a naturally occurring molecule or may be a synthetic compound, including, for example and not for limitation, a small molecule (e.g., a molecule having a molecular weight<1000 Daltons), a peptide, a protein, an antibody, or a nucleic acid, used to treat an individual with AAA or other disorder of the vascular system.

“Progression” refers to an increase in symptoms of AAA, including, for example and not for limitation, increased stretching of the aortic wall, decreased vascular function, or increased pain or internal bleeding, for an individual with AAA undergoing examination or treatment for the disorder.

### II. Biomarkers

Biomolecules present in the blood, plasma, serum, urine or other body fluid, or in a tissue sample, may be present at different levels in individuals with a disorder or condition as compared to otherwise healthy individuals or a control population. The inventor has discovered that particular proteins are present in the serum, plasma or urine of individuals with AAA at elevated or reduced levels compared to age-matched control individuals.

In one aspect, the invention relates to biomolecules, in particular, proteins, that are differentially present in serum, plasma or urine from individuals with AAA as compared to age-matched control individuals (i.e., individuals without the disorder). These proteins are therefore associated with AAA and are termed AAA-associated proteins (biomarkers). These biomarkers are present in individuals with AAA at either elevated or reduced levels compared to healthy individuals. Exemplary biomarkers shown to be present in individuals with AAA at different levels compared to age-matched control individuals are provided in Tables 1-4, as described in Examples 5-7.

The biomarkers of the invention are proteins identified and characterized by their body fluid source, their binding characteristics to adsorbant surfaces of a SELDI probe, their mass-to-charge ratio as determined by mass spectrometry, and the shape of the spectral peak in time-of-flight mass spectrometry (ToF-MS). These characteristics provide one method of uniquely identifying biomolecules and determining whether a biomolecule is a biomarker of the invention. These characteristics represent inherent properties of biomolecules and not process limitations in the manner in which the biomolecules are discriminated.

As discussed in detail in the Examples, the biomarkers of the invention were identified using SELDI technology employing PROTEINCHIP arrays from Ciphergen Biosystems, Inc. (Fremont, Calif.). Serum, plasma or urine samples were collected from individuals diagnosed with AAA and from age-matched control individuals not diagnosed with AAAA. In some circumstances, the serum, plasma or urine samples were pre-fractionated by albumin and IgG depletion (see, e.g., Examples 2 and 6, infra). In other circumstances, the serum, plasma or urine samples were used without prior fractionation. Samples, either fractionated or not, were applied to SELDI biochips and the spectra of proteins in the samples that bound to the biochips were generated by ToF-MS on a Ciphergen PBSII mass spectrometer. The spectra were analyzed by Ciphergen Express Data Manager Software with BIOMARKER WIZARD and Biomarker Pattern Software from Ciphergen Biosystems, Inc. The mass spectra for each group were subjected to scatter plot analysis. A Student's t-test analysis was employed to compare AAA and control groups for each protein cluster in the scatter plot, and proteins were selected that differed significantly (p-value<0.05, or, in some cases, <0.1) between the two groups. This method is described in more detail in Example 5.

In the practice of the invention, biomarkers can be obtained in a biological sample, preferably a fluid sample, of the individual. The biomarkers are preferentially obtained in a sample of the individual's blood, serum, plasma, urine, CSF or saliva. The biological sample can also be a tissue sample, e.g., a skin biopsy. The biological sample can be depleted of particular proteins, for example, albumin and IgG, if appropriate.

In one embodiment, a method for diagnosing AAA, assessing the efficacy of preventive treatment for AAA, or monitoring the progression of AAA, in an individual involves obtaining a sample of blood, serum or plasma from the individual and determining the levels of at least two biomarkers. As an example, the biomarkers of the invention were obtained from the serum, plasma and blood of individuals with AAA and age-matched control individuals, as described in Examples 5-7.

Examples of identified and characterized biomarkers for diagnosing AAA, assessing the efficacy of preventive treatment for AAA, or monitoring the progression of AAA are presented in Tables 1-4. The “Mass” column refers to the mass-to-charge ratio in Daltons (Da) as determined by mass spectrometry. The “Assay” column refers to the type of SELDI biochip used bind the biomarker, the chromatographic fraction and/or wash condition used, if applicable, and the mass spectrometry condition (i.e., type of matrix and laser setting), as described in detail in the Examples. The “P-value” column refers to the statistical significance reached for the difference in the level of the indicated biomarker between samples from individuals diagnosed with AAA and control individuals. The “Up/Down” column specifies whether the level of the indicated biomarker is elevated or reduced in individuals diagnosed with AAA as compared to control individuals.

The biomarkers of the invention are identified and characterized by their mass-to-charge ratio as determined by mass spectrometry. The mass-to-charge ratio of each biomarker is provided in the “Mass” column in Tables 1-4. For example, the mass-to-charge ratio of protein #2 in Table 1 is 2685. The mass-to-charge ratios were determined by mass spectra generated on a Ciphergen Biosystems, Inc. PBS II mass spectrometer. This instrument has a mass accuracy of about +/−0.15 percent (e.g., for a 5,000 Da protein, the error is ±7.5 Da). Thus, the biomarkers herein which are referred to by a measured apparent mass are not expected to provide precisely the same apparent mass every time their presence is detected in a given sample. Additionally, the PBS II mass spectrometer has a mass resolution of about 400 to 1000 m/dm, where m is mass and dm is the mass spectral peak width at 0.5 peak height. The mass-to-charge ratio of the biomarkers was determined using BIOMARKER WIZARD software (Ciphergen Biosystems, Inc.). BIOMARKER WIZARD assigns a mass-to-charge ratio to a biomarker by clustering the mass-to-charge ratios of the same peaks from all the spectra analyzed, as determined by the PBS II mass spectrometer, taking the maximum and minimum mass-to-charge-ratio in the cluster, and dividing by two. Accordingly, the masses provided reflect these specifications.

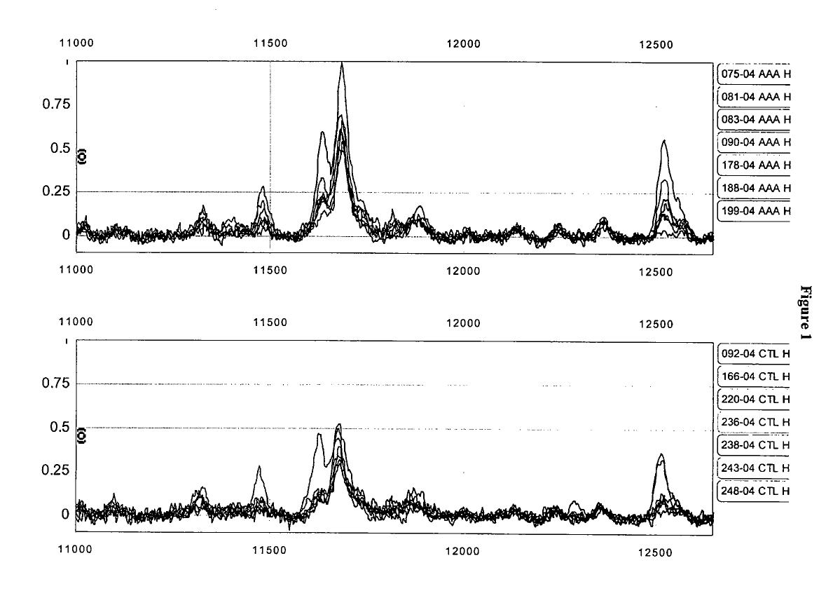

The biomarkers of the invention are also characterized by the shape of their spectral peak in ToF-MS. A representative example of mass spectra showing peaks representing potential biomarkers of the invention is presented in FIG. 1.

The biomarkers of the invention are also characterized by the source of biological sample and chromatographic fraction, if appropriate, in which the biomarker is found. Examples of biological samples containing the biomarkers of the invention include, for example and not for limitation, serum, plasma and urine. Examples of chromatographic fractions containing the biomarkers of the invention include, for example and not for limitation, anion exchange chromatography fraction, cation exchange chromatography fraction, and size exclusion chromatographic fraction.

The biomarkers of the invention are also characterized by their binding properties on chromatographic surfaces. The biomarkers of the invention bind to cation exchange adsorbants (e.g., CM10 or WCX2 PROTEINCHIP array from Ciphergen Biosystems, Inc.), anion exchange adsorbants (e.g., SAX2 or Q10 PROTEINCHIP array from Ciphergen Biosystems, Inc.), hydrophobic exchange adsorbants (e.g., H4 or HSO PROTEINCHIP array from Ciphergen Biosystems, Inc.), hydrophilic exchange adsorbants (e.g., NP20 PROTEINCHIP from Ciphergen Biosystems, Inc.) and/or immobilized metal affinity capture (IMAC) adsorbants (e.g., IMAC3 or IMAC30 PROTEINCHIP array from Ciphergen Biosystems, Inc.).

The biomarkers of this invention are characterized by their charge-to-mass ratio, the shape of their spectral peaks, the source of biological sample and chromatographic fraction, and their binding properties on chromatographic surfaces. Thus, a biomarker of the invention can be uniquely identified without knowledge of its specific molecular identity. However, if desired, the specific molecular identity of a biomarker of the invention can be determined by, for example, determining the amino acid sequence of the protein, e.g., by peptide mapping or sequencing. For example, a biomarker can be peptide mapped using a number of proteases, such as trypsin or V8 protease, and the molecular weights of the resultant peptide digestion fragments can be used to search databases for sequences that match the molecular weights of the digestion fragments generated by the various proteases. Alternatively, biomarkers can be sequenced using tandem MS. In this method, the protein is isolated, for example, by gel electrophoresis. The protein is excised from the gel and subjected to proteolytic digestion. Individual peptide fragments are separated by MS, subjected to collision-induced cooling, further fragmenting the peptides and producing a polypeptide ladder, which is then analyzed by MS. The difference in mass of the members of the polypeptide ladder identifies the amino acids in the sequence. This method can be used to determine the entire protein sequence, or to use a sequenced peptide fragment to search databases to for matching sequences.

Once the sequence of a biomarker of the invention is determined, its presence in a biological sample from an individual can be measured by methods known in the art, including for example and not for limitation methods described in paragraph [0053] above.

The biomarkers of the invention can be detected in the serum of an individual. The biomarkers of the invention can be detected in other blood fractions, i.e., plasma, or in urine. Many of the biomarkers of the invention can be found in both serum and plasma, and in urine. The biological sample can be depleted of albumin and IgG, if appropriate.

The biomarkers of the invention are biomolecules. Accordingly, this invention provides these biomolecules in isolated form. The biomarkers can be isolated from biological fluids, such as, for example, serum, plasma and urine. They can be isolated by any method known in the art, based on both their mass and/or their binding characteristics. For example, a sample comprising the biomolecules can be subject to chromatographic fractionation, as described herein, and subject to further separation by, e.g., acrylamide gel electrophoresis. The determination of the molecular identity of the biomarker also allows their isolation by immunoaffinity chromatography.

The biomarkers of the invention can exist in a biological sample from an individual in various forms with different mass-to-charges ratios. For example, different forms of biomarkers can result from either pre- or post-translational modification, or both. Pre-translational modifications include, for example, allelic variants and splice variants. Post-translation modifications include, for example, proteolytic cleavage, glycosylation, phopshorylation, lipidation, oxidation, methylation, cystinylation, sulphonation and acetylation. Once the sequence of a biomarker of the invention is determined, the presence and level of various forms of the biomarker in a biological sample from an individual can be determined by methods known in the art, as described above. In certain cases, a modified form of the biomarker may have a more pronounced difference in expression between individuals diagnosed with AAA and control individuals than its unmodified form.

III. Detection of Biomarkers Associated with AAA

AAA biomarkers can be separated and detected using any of a number of methods including immunological assays (e.g., ELISA), separation-based methods (e.g., gel electrophoresis), protein-based methods (e.g., mass spectroscopy), function-based methods (e.g., enzymatic or binding activity), and the like. Other methods will be known to those of skill in the art guided by this specification. The method used for detecting the biomarkers and determining their levels will depend, in part, on the identity and nature of the biomarker protein. Suitable methods for detecting the biomarkers of the invention include, for example and not for limitation, optical methods, including confocal and non-confocal microscopy, and detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), electrochemical methods (e.g., voltametry and amperometry techniques), atomic force microscopy, and radio frequency methods (e.g., multipolar resonance spectroscopy).

In one embodiment, the method for separating, detecting and determining the levels of at least two biomarkers of the invention involves obtaining a biological sample from an individual, separating the proteins by chromatography, if appropriate, capturing the proteins on a biochip (i.e., an adsorbent of a SELDI probe), and detecting and determining the levels of the captured biomarkers by mass spectrometry (i.e., ToF-MS).

A biochip generally comprises a solid substrate and has a generally planar surface to which a capture reagent (also called an adsorbent or affinity reagent) is attached. Frequently, the surface of a biochip comprises a plurality of addressable locations, each of which has the capture reagent bound thereto.

A “protein biochip” refers to a biochip adapted for the capture of proteins. Protein biochips are known in the art, including, for example, those produced by Ciphergen Biosystems, Inc. (Fremont, Calif.), Packard BioScience Company (Meriden, Conn.), Zyomyx (Hayward, Calif.), Phylos (Lexington, Mass.) and Biacore (Uppsala, Sweden). Examples of such protein biochips are described in, e.g., U.S. Pat. Nos. 6,225,047, 6,329,209 and 5,242,828, and PCT Publication Nos. WO 99/51773 and WO 00/56934.

In one embodiment, the biomarkers of the invention are detected by mass spectrometry (MS) methods. Examples of mass spectrometers are time-of-flight (ToF), magnetic sector, quadrupole filter, ion trap, ion cyclotron resonance, electrostatic sector analyzer, and hybrids of these.

In one embodiment, the mass spectrometer is a laser desorption/ionization mass spectrometer. In laser desorption/ionization mass spectrometry, the analytes (i.e., proteins) are placed on the surface of a MS probe, which engages a probe interface of the mass spectrometer and presents an analyte to ionizing energy for ionization and introduction into the mass spectrometer. A laser desorption mass spectrometer employs laser energy, typically from an ultraviolet laser, but also from an infrared laser, which desorbs the analytes from the surface, and volatilizes and ionizes the analytes, thereby making them available to the ion optics of the mass spectrometer.

A mass spectrometry method for use in the invention is “Surface Enhanced Laser Desorption and Ionization” or “SELDI,” as described, for example, in U.S. Pat. Nos. 5,719,060 and 6,225,047. SELDI refers to a method of desorption/ionization gas phase ion spectrometry in which the analyte (i.e., at least two of the biomarkers) is captured on the surface of a SELDI MS probe. There are several versions of SELDI, including “affinity capture mass spectrometry,” “Surface-Enhanced Affinity Capture” or “SEAC,” “Surface-Enhanced Neat Desorption” or “SEND,” and “Surface-Enhanced Photolabile Attachment and Release” or “SEPAR”.

SEAC involves the use of probes having a material on the probe surface that captures analytes (i.e., proteins) through non-covalent affinity interactions (i.e., adsorption) between the material and the analyte. The material is variously called an “adsorbent,” a “capture reagent,” an “affinity reagent” or a “binding moiety.” Such probes are called “affinity capture probes” having “adsorbent surfaces.” The capture reagent can be any material capable of binding an analyte. The capture reagent may be attached directly to the substrate of the selective surface, or the substrate may have a reactive surface that carries a reactive moiety capable of binding the capture reagent, e.g., through a reaction forming a covalent or coordinate covalent bond. Epoxide and carbodiimidizole are useful reactive moieties to covalently bind protein capture reagents, such as antibodies or cellular receptors. Nitriloacetic acid and iminodiacetic acid are useful reactive moieties that function as chelating agents to bind metal ions that interact non-covalently with histidine containing peptides. Adsorbents are generally classified as either chromatographic adsorbents or biospecific adsorbents.

A “chromatographic adsorbent” refers to an adsorbent material typically used in chromatography. Chromatographic adsorbents include, for example, anion and cation exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

A “biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, or a nucleic acid (e.g., DNA)-protein conjugate). In certain instances, the biospecific adsorbent can be a macromolecular structure, such as a multi-protein complex, a biological membrane or a virus. Examples of biospecific adsorbents include antibodies, receptor proteins and nucleic acids. Typically, biospecific adsorbents have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity typically of at least 10⁻⁸ M.

Protein biochips produced by Ciphergen Biosystems, Inc. comprise surfaces having chromatographic or biospecific adsorbents attached thereto at addressable locations. Ciphergen PROTEINCHIP arrays include NP20 (hydrophilic); H4 and H50 (hydrophobic); SAX2, Q10 and LSAX30 (anion exchange); WCX2, CM10 and LWCX30 (cation exchange); IMAC3, IMAC30 and IMAC40 (metal chelate); and PS10, PS20 (reactive surface with carboimidizole, expoxide) and PG20 (protein G coupled through carboimidizole). Hydrophobic PROTEINCHIP arrays have isopropyl or nonylphenoxy-poly(ethylene glycol)methacrylate functionalities. Anion exchange PROTEINCHIP arrays have quaternary ammonium functionalities. Cation exchange PROTEINCHIP arrays have carboxylate functionalities. Immobilized metal chelate PROTEINCHIP arrays have nitriloacetic acid functionalities that adsorb transition metal ions, such as copper, nickel, zinc, and gallium, by chelation. Preactivated PROTEINCHIP arrays have carboimidizole or epoxide functional groups that can react with groups on proteins for covalent binding.

Protein biochips are further described in U.S. Pat. Nos. 6,579,719 and 6,555,813, PCT Publication Nos. WO 00/66265 and WO 03/040700, U.S. Patent Application Nos. US 20030032043 A1, US 20030218130 A1 and US 20050059086 A1.

In general, a probe with an adsorbent surface is contacted with the sample for a period of time sufficient to allow proteins present in the sample to bind to the adsorbent. After the incubation period, the substrate is washed to remove unbound material. Any suitable washing solutions can be used; preferably, aqueous solutions are employed. The extent to which proteins remain bound to the adsorbent can be manipulated by adjusting the stringency of the wash. The elution characteristics of a wash solution can depend, for example, on pH, ionic strength, hydrophobicity, degree of chaotropism, detergent strength, temperature, and the like. Unless the probe has both SEAC and SEND properties (as described herein), an energy absorbing molecule is then applied to the substrate with the bound proteins.

The biomarkers bound to the substrates are detected in a gas phase ion spectrometer such as a ToF mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically involves detection of signal intensity. Thus, both the quantity and mass of the biomarker can be determined.

SEND involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. In certain embodiments, the EAM are incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 6,124,137 and PCT Publication No. WO 03/64594.

SEAC/SEND is a version of SELDI in which both a capture reagent and an EAM are attached to the sample presenting surface. SEAC/SEND probes therefore allow the capture of analytes through affinity capture and ionization/desorption without the need to apply an external matrix. The C18 SEND biochip is a version of SEAC/SEND, comprising a C18 moiety which functions as a capture reagent, and a CHCA moiety which functions as an EAM.

SEPAR involves the use of probes having moieties attached to the surface that can covalently bind an analyte, and then release the analyte through breaking a photolabile bond in the moiety after exposure to light, e.g., to laser light (see U.S. Pat. No. 5,719,060). SEPAR and other forms of SELDI are readily adapted to detecting a biomarker or biomarker profile, pursuant to the present invention.

In another MS method, the biomarkers are first captured on a resin having chromatographic properties that bind biomarkers. In the examples herein, this could include a variety of methods. For example, one could capture the biomarkers on a cation exchange resin, such as CM CERAMIC HYPERD F resin, wash the resin, elute the biomarkers and detect them by MALDI. Alternatively, this method could be preceded by fractionating the sample on an anion exchange resin, such as Q CERAMIC HYPERD F resin, before application to the cation exchange resin. In another alternative, one could fractionate the sample on an anion exchange resin and detect by MALDI directly. In yet another method, one could capture the biomarkers on an immuno-chromatographic resin comprising antibodies that bind particular biomarkers, wash the resin to remove unbound material, elute the biomarkers from the resin and detect the eluted biomarkers by MALDI or by SELDI.

Analysis of analytes by ToF-MS generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing using Ciphergen's PROTEINCHIP software, or any equivalent data processing software. Data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass-to-charge ratio information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, for example, as part of Ciphergen's PROTEINCHIP software package, which can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of at least two peaks, the shape of a peak or group of peaks, the height of at least two peaks, the log of the height of at least two peaks, and other arithmetic manipulations of peak height data.

A general protocol for the detection of biomarkers of the invention is as follows. The biological sample to be tested is obtained from consenting individuals diagnosed with AAA and control individuals, depleted of albumin and IgG or pre-fractionated on an anion exchange resin or other chromatographic resin, as appropriate, and then contacted with an affinity capture SELDI probe comprising a cation exchange adsorbant (e.g., CM10 or WCX2 PROTEINCHIP array from Ciphergen Systems, Inc.), an anion exchange adsorbant (e.g., Q10 PROTEINCHIP array from Ciphergen Systems, Inc.), a hydrophobic exchange adsorbant (e.g., HSO PROTEINCHIP array from Ciphergen Systems, Inc.), or an IMAC adsorbant (e.g., IMAC3 or IMAC30 PROTEINCHIP array from Ciphergen Systems, Inc.). The SELDI probe is washed with a suitable buffer that retains the biomarkers of the invention, while washing away unbound biomolecules. Examples of suitable buffers are described in Examples 2-3. The biomarkers specifically retained on the SELDI probe are then detected by laser desorption/ionization mass spectrometry.

The biological sample, e.g., serum, plasma or urine, can be depleted of albumin and IgG or subjected to pre-fractionation before binding to a SELDI probe. One method of pre-fractionation involves contacting the biological sample with an anion exchange chromatographic resin. The bound biomolecules are then subjected to stepwise pH elution using buffers at various pH, as described in the Examples. Various fractions containing biomolecules are collected and subjected to binding to a SELDI probe.

Alternatively, if analysis of particular proteins and various forms thereof is desired, antibodies which recognize specific proteins can be attached to the surface of a SELDI probe (e.g., pre-activated P510 or PS20 PROTEINCHIP array from Ciphergen Systems, Inc.). The antibodies capture the target proteins from a biological sample onto the SELDI probe. The captured proteins are then detected by, e.g., laser desorption/ionization mass spectrometry. The antibodies can also capture the target proteins on immobilized support, and the target proteins can be eluted and captured on a SELDI probe and detected as described above.

Antibodies to target proteins are either commercially available or can be produced by methods known in the art, e.g., by immunizing animals with the target proteins isolated by standard purification techniques or with synthetic peptides of the target proteins.

In some cases it will be desirable to establish normal or baseline values (or ranges) for biomarker expression levels. Normal levels can be determined for any particular population, subpopulation, or group of humans according to standard methods well known to those of skill in the art. Generally, baseline (normal) levels of biomarkers are determined by quantifying the amount of a biomarker in biological samples (e.g., fluids, cells or tissues) obtained from normal (healthy) subjects. Application of standard statistical methods used in medicine permits determination of baseline levels of expression, as well as significant deviations from such baseline levels.

In carrying out the diagnostic and prognostic methods of the invention, as described above, it will sometimes be useful to refer to “diagnostic” and “prognostic” values. As used herein, “diagnostic value” refers to a value that is determined for the biomarker gene product detected in a sample which, when compared to a normal (or “baseline”) range of the biomarker gene product is indicative of the presence of a disorder. “Prognostic value” refers to an amount of the biomarker that is consistent with a particular diagnosis and prognosis for the disorder. The amount of the biomarker gene product detected in a sample is compared to the prognostic value for the biomarker such that the relative comparison of the values indicates the presence of the disorder or the likely outcome of the disorder. In one embodiment, for example, to assess AAA prognosis, data are collected to obtain a statistically significant correlation of biomarker levels with different degrees of severity of AAA (e.g., size or diameter of aneurysm). A predetermined range of biomarker levels is established from subjects having known clinical outcomes. A sufficient number of measurements is made to produce a statistically significant value (or range of values) to which a comparison will be made.

It will be appreciated that the assay methods do not necessarily require measurement of absolute values of a biomarker, unless it is so desired, because relative values are sufficient for many applications of the methods of the present invention. Where quantification is desirable, the present invention provides reagents such that virtually any known method for quantifying gene products can be used.

### IV. Diagnosis of AAA

In a first aspect, the invention provides a method for diagnosing AAA in a individual, by determining the levels of at least two, at least 3, at least 4, at least 5, at least 6, at least 7, at least 8, at least 9 or at least 10 biomarkers in a sample from the individual, and comparing the levels of the biomarkers in the sample from the individual to reference levels of the biomarkers characteristic of a control population, where a difference in the levels of the biomarkers between the sample from the individual and the control population indicates that the individual has AAA. The methods include obtaining a sample from the individual and determining the levels of the biomarkers. The levels of certain biomarkers are significantly different in individuals with AAA than in healthy individuals. The levels of certain biomarkers are higher in individuals with AAA than in healthy individuals. The levels of certain biomarkers are lower in individuals with AAA than in healthy individuals. The biomarkers can be obtained in a biological sample, and the levels of the at least two biomarkers can be determined, by any suitable method, as described above.

As used herein, the term “diagnosis” is not limited to a definitive or near definitive determination that an individual has a disorder, but also includes determining that an individual has an increased likelihood of having or increased propensity for developing the disorder, compared to healthy individuals or to the general population. For example, a patient with very early asymtomatic disease or disease antecedents may be identified. The methods of the invention may be used for screening.

In one embodiment, a method for diagnosing AAA in a individual involves obtaining a biological sample from the individual and determining the levels of the at least two biomarkers by fractionating the biomarkers in the biological sample on an anion exchange chromatographic resin, binding the biomarkers to a SELDI probe, and detecting the bound biomarkers by laser desorption/ionization mass spectrometry.

In one embodiment, a method for diagnosing AAA in a individual involves obtaining a biological sample from the individual and determining the levels of the at least two biomarkers by binding the biomarkers to a SELDI probe, and detecting the bound biomarkers by laser desorption/ionization mass spectrometry.

In one embodiment, a method for diagnosing AAA in a individual involves obtaining a sample of blood, serum, plasma or urine from the individual and determining the levels of the at least two biomarkers. The biological sample can be depleted of albumin and IgG, if appropriate.

In one embodiment, the method for diagnosing AAA involves determining the level of one biomarker. An example of a single biomarker that may be used is protein #2 (mass-to-charge ratio 2685) as shown in Table 1 in Example 5.

In one embodiment, the method for diagnosing AAA involves determining the levels of more than one biomarker. Examples of combinations or sets of biomarkers are described in paragraphs [0021] to [0032].

In one embodiment, the method for diagnosing AAA involves determining the levels of a set of biomarkers (i.e., more than one biomarker). The biomarkers in a particular set may be related or grouped in a number of ways. By measuring multiple biomarkers, conclusions can be reached that are more precise and with higher confidence. The biomarkers in a set may be related by the magnitude of the difference in their levels between individuals diagnosed with AAA and control individuals. For example, in one embodiment the levels of biomarkers in controls compared to individuals diagnosed with AAA differs by a factor of at least 1.5-fold, sometime at least 2-fold and sometimes at least 2.5-fold. Other sets include biomarkers having an at least 1.25-fold, at least 3-fold, at least 4-fold, at least 5-fold, or at least 10-fold difference between individuals diagnosed with AAA and control individuals. In one embodiment, biomarkers in a set are related by the direction of change in individuals diagnosed with AAA compared to controls, i.e., at elevated or reduced levels, indicated as “Up” or “Down”, respectively, in Tables 1-4.

In one embodiment, the method for diagnosing AAA involves determining the levels of a set of biomarkers (i.e., more than one biomarker) in which all of the biomarkers in the set are present at elevated levels in individuals diagnosed with AAA as compared to control individuals. Examples of such a set of biomarkers are provided in Tables 1-4 below (biomarkers indicated as “Up” in the “Up/Down” column). In one embodiment, the set of biomarkers can comprise at least 2, at least 3, at least 4, or at least 5 of the biomarkers listed as “Up” in Tables 1-4.

In one embodiment, the method for diagnosing AAA involves determining the levels of a set of biomarkers (i.e., more than one biomarker) in which all of the biomarkers in the set are present at reduced levels in individuals diagnosed with AAA as compared to control individuals. An example of such a set of biomarkers is provided in Tables 1-4 below (biomarkers indicated as “Down” in the “Up/Down” column). In one embodiment, the set of biomarkers can comprise at least 2, at least 3, at least 4, or at least 5 of the biomarkers listed as “Down” in Tables 1-4.

In one embodiment, the method for diagnosing AAA involves determining the levels of a set of biomarkers such as, without limitation, those sets described in Section VIII below.

### V. Assessing the Efficacy of Preventive Treatment for AAA

In a first aspect, the invention provides a method for assessing the efficacy of preventive treatment for AAA in an individual, comprising determining the levels of at least two biomarkers in a sample from the individual before treatment or at a first time point after treatment, and determining the levels of the at least two biomarkers in the individual at a later time point or time points during treatment or after treatment, and comparing the levels of the at least two biomarkers at the two or more time points. A change from a level characteristic of AAA to a more normal level is an indication of efficacy of the preventive treatment. The methods include obtaining a sample from the individual and determining the levels of the at least two biomarkers. The levels of certain biomarkers are higher in individuals with AAA than in healthy individuals. The levels of these biomarkers in an individual with AAA decrease upon treatment with an agent effective to treat AAA. The levels of certain other biomarkers are lower in individuals with AAA than in healthy individuals. The levels of these biomarkers in an individual with AAA increase upon treatment with an agent effective to prevent or delay AAA. The methods include obtaining a sample from the individual and determining the levels of the at least two biomarkers as above.

In one embodiment, a method for assessing the efficacy of preventive treatment of AAA in an individual involves the individual being treated with an agent effective to prevent or delay the disorder.

In one embodiment, a method for assessing the efficacy of preventive treatment of AAA in a individual involves obtaining a biological sample from the individual and determining the levels of the at least two biomarkers by fractionating the biomarkers in the biological sample on an anion exchange chromatographic resin, binding the biomarkers to a SELDI probe, and detecting the bound biomarkers by laser desorption/ionization mass spectrometry.

In one embodiment, a method for assessing the efficacy of preventive treatment of AAA in a individual involves obtaining a biological sample from the individual and determining the levels of the at least two biomarkers by binding the biomarkers to a SELDI probe, and detecting the bound biomarkers by laser desorption/ionization mass spectrometry.

In one embodiment, a method for assessing the efficacy of preventive treatment of AAA in a individual involves obtaining a sample of blood, serum, plasma or urine from the individual and determining the levels of the at least two biomarkers. The biological sample can be depleted of albumin and IgG, if appropriate.

In one embodiment, the method for assessing the efficacy of preventive treatment of AAA involves determining the level of one biomarker. An example of a single biomarker that may be used is protein #2 (mass-to-charge ratio 2685) as shown in Table 1 in Example 5.

In one embodiment, the method for assessing the efficacy of preventive treatment of AAA involves determining the levels of more than one biomarker. Examples of combinations or sets of biomarkers are described in paragraphs [0021] to [0032].

In one embodiment, the method for assessing the efficacy of preventive treatment of AAA involves determining the levels of a set of biomarkers (i.e., more than one biomarker). The biomarkers in a particular set may be related or grouped in a number of ways. By measuring multiple biomarkers, conclusions can be reached that are more precise and with higher confidence. The biomarkers in a set may be related by the magnitude of the difference in their levels between individuals diagnosed with AAA and control individuals. For example, in one embodiment the levels of biomarkers in controls compared to individuals diagnosed with AAA differs by a factor of at least 1.5-fold, sometime at least 2-fold and sometimes at least 2.5-fold. Other sets include biomarkers having an at least 1.25-fold, at least 3-fold, at least 4-fold, at least 5-fold, or at least 10-fold difference between individuals diagnosed with AAA and control individuals. In one embodiment, biomarkers in a set are related by the direction of change in individuals diagnosed with AAA compared to controls, i.e., at elevated or reduced levels, indicated as “Up” or “Down”, respectively, in Tables 1-4.

In one embodiment, the method for assessing the efficacy of preventive treatment of AAA involves determining the levels of a set of biomarkers (i.e., more than one biomarker) in which all of the biomarkers in the set are present at elevated levels in individuals diagnosed with AAA as compared to control individuals. Examples of such a set of biomarkers are provided in Tables 1-4 below (biomarkers indicated as “Up” in the “Up/Down” column). In one embodiment, the set of biomarkers can comprise at least 2, at least 3, at least 4, or at least 5 of the biomarkers listed as “Up” in Tables 1-4.

In one embodiment, the method for assessing the efficacy of preventive treatment of AAA involves determining the levels of a set of biomarkers (i.e., more than one biomarker) in which all of the biomarkers in the set are present at reduced levels in individuals diagnosed with AAA as compared to control individuals. An example of such a set of biomarkers is provided in Tables 1-4 below (biomarkers indicated as “Down” in the “Up/Down” column). In one embodiment, the set of biomarkers can comprise at least 2, at least 3, at least 4, or at least 5 of the biomarkers listed as “Down” in Tables 1-4.

In one embodiment, the method for assessing the efficacy of preventive treatment of AAA involves determining the levels of a set of biomarkers such as, without limitation, those sets described in Section VIII below.

### VI. Monitoring Progression of AAA

In one aspect, the invention provides a method for monitoring the progression of AAA, comprising detecting one of more biomarkers in a sample from the individual. In one embodiment, the individual is under treatment with an agent effective to treat or prevent AAA, and the levels of the at least two biomarkers determine the future treatment regime for the individual. The methods include obtaining a sample from the individual and determining the levels of the at least two biomarkers as above.

In one embodiment, a method for monitoring the progression of AAA in a individual involves obtaining a biological sample from the individual and determining the levels of the at least two biomarkers by fractionating the biomarkers in the biological sample on an anion exchange chromatographic resin, binding the biomarkers to a SELDI probe, and detecting the bound biomarkers by laser desorption/ionization mass spectrometry.

In one embodiment, a method for monitoring the progression of AAA in a individual involves obtaining a biological sample from the individual and determining the levels of the at least two biomarkers by binding the biomarkers to a SELDI probe, and detecting the bound biomarkers by laser desorption/ionization mass spectrometry.

In one embodiment, a method for monitoring the progression of AAA in a individual involves obtaining a sample of blood, serum, plasma or urine from the individual and determining the levels of the at least two biomarkers. The biological sample can be depleted of albumin and IgG, if appropriate.

In one embodiment, the method for monitoring the progression of AAA involves determining the level of one biomarker. An example of a single biomarker that may be used is protein #2 (mass-to-charge ratio 2685) as shown in Table 1 in Example 5.

In one embodiment, the method for monitoring the progression of AAA involves determining the levels of more than one biomarker. Examples of combinations or sets of biomarkers are described in paragraphs [0021] to [0032].

In one embodiment, the method for monitoring the progression of AAA involves determining the levels of a set of biomarkers (i.e., more than one biomarker). Sets may be defined, for example, as described above.

In one embodiment, the method for monitoring the progression of AAA involves determining the levels of a set of biomarkers such as, without limitation, those sets described in Section VIII below.

### VII. Kits

In one aspect, the invention provides a kit comprising a solid support comprising at least one capture reagent attached thereto, wherein the capture reagent binds at least two biomarkers selected from the biomarkers indicated in Tables 1-4, at least two biomarkers selected from the biomarkers indicated in Tables 1-4, and instructions for using the solid support to detect the biomarkers selected from the biomarkers indicated in Tables 1-4.

In various embodiments, the kit contains a combination or set of biomarkers, for example, as described in paragraphs [0021] to [0032] above.

### VIII. Exemplary Sets of Biomarkers

The invention provides methods for diagnosing abdominal aortic aneurysm (AAA) (see Section IV, supra), for assessing the efficacy of preventive treatment of AAA (see Section V, supra), and for monitoring the progression of AAA (see Section VI, supra), in an individual. The invention also provides kits useful for diagnosing AAA, assessing the efficacy of preventive treatment of AAA, and monitoring the progression of AAA (see Section VII, supra). The methods and kits use at least 1, at least 2, at least 3, at least 4, at least 5, at least 6, at least 7, at least 8, at least 9, or at least 10 biomarkers that are associated with AAA listed in Tables 1-4.

The experiments described in the Examples below were used to identify and characterize the biomarkers of the invention.

### Example 1

### Collection and Preparation of Serum, Plasma and Urine Samples

The following protocols are used to collect and prepare the biological samples obtained from individuals to identify and characterize the biomarkers of the invention.

Serum and Plasma. Blood is drawn from individuals into one tube each of Grey Top (sodium fluoride/potassium oxalate) and Red Top (empty) to prepare plasma and serum, respectively. The tubes are stored upright in a refrigerator until ready for processing. A preferred storage time is less than 1 hour. Blood in the Red Top tubes is allowed to coagulate for 1 hour at room temperature (RT), and then is centrifuged at 1500 g for 10 min at RT. The supernatant is aspirated into separate tubes and centrifuged again at 3000 g for 10 min at RT. The resulting supernatant is divided into following aliquots: 4×254, 2×1004, and 2×250 μL in Eppendorf tubes. The remaining supernatant is divided in 500 μL aliquots. This aliquot scheme can be modified depending on whether native serum or plasma is used, or if depleted or pre-fractionated serum or plasma is used. All tubes are labeled, flash frozen in LN₂ and stored at −80° C.

Urine. Urine is obtained from individuals and centrifuged at 16,000 g for 10 min at 4° C., and the supernatant is aliquoted into 10×500 μL Eppendorf tubes. The remainder of the supernatant is divided into 15 mL culture tubes. All tubes are frozen at −80° C. until analysis.

### Example 2

### Processing of Serum, Plasma and Urine Samples

The following protocols are used to process the serum, plasma and urine samples obtained from individuals to identify and characterize the biomarkers of the invention.

The required number of aliquots are thawed at RT and centrifuged at 10,000 g for 2 min at RT. The supernatant is aspirated into separate tubes for further processing.

Native Serum or Plasma. Serum or plasma samples are denatured by diluting 1:5 in extraction buffer (9M Urea, 2% CHAPS, 2.3% DTT, 50 mM Tris-HCl pH 9) (10 μL serum or plasma+40 μL extraction buffer) and incubating for 30 minutes at RT with shaking. Alternatively, serum or plasma samples are denatured by diluting 1:5 in Hepes buffer (10 μL serum+40 μL buffer). A portion of the diluted, denatured serum or plasma samples are further diluted 1:20 in Hepes buffer (5 μL of 1:5 dilution+100 μL Hepes buffer) to yield a final 1:100 diluted serum or plasma sample to be used for protein determination. The 1:5 diluted, denatured serum or plasma samples are further diluted 1:5 in various buffers (1:25 total dilution) for binding onto different biochip surfaces under different binding conditions.

Albumin and IgG Depletion of Serum. Undiluted serum or plasma is used for albumin and IgG depletion using the Aurum Serum Protein Mini Kit (Bio-Rad). A spin column is filled with Affi-Gel Blue (to bind Albumin) and Affi-Gel Protein A (to bind IgG) resins, and placed in a 12×75 mm test tube. The resins are allowed to settle for at least 5 min. The tip is broken off the bottom of the column, the cap removed, and the column is placed back in the test tube. Residual buffer is drained from the column by gravity flow. The columns is washed twice with 1 ml of protein-binding buffer supplied with the kit. The column is drained completely each time. The column is placed in a 2 ml collection tube and centrifuged for 20 seconds at 10,000 g to dry the resins. A yellow column tip is placed on the bottom of the column to stop flow, and the column is placed in a clean 2 ml collection tubes labeled “unbound”. In a separate tube, 60 μL of serum or plasma is mixed thoroughly with 180 μL of protein-binding buffer, and 200 μL of the diluted sample is added to the top of the resins bed. After allowing the samples to penetrate the resins, the column is gently vortexed. The vortexing step is repeated at 5 min and 10 min. The column is allowed to sit for another 5 min. The yellow tip is removed, and the column is centrifuged for 20 sec at 10,000 g. The eluate was collected in the 2 ml tube labeled “unbound”. The resins are washed with 200 μL of binding buffer, vortexed gently and centrifuged for 20 sec at 10,000 g. The eluate was collected in the same tube as above. This tube now contains 400 μL of albumin- and IgG-depleted samples. The yield should be about 1.5 to 2 mg/ml protein. The bound albumin and IgG can be recovered from the column. For 1-D analysis, the column can be eluted with 500 μL of Laemmli sample buffer (62.5 mM Tris-Hcl pH 6.8; 10% glycerol; 2% SDS; 1 mg/ml DTT; 0.05% Bromophenol Blue). For 2-D analysis, the column can be eluted with 500 μL of ReadyPrep sequential extraction reagent 3 (Bio-Rad). The albumin- and IgG-depleted serum is denatured by diluting 2× in extraction buffer (9M Urea, 2% CHAPS, 2.3% DTT, 50 mM Tris-HCl pH 9) (e.g., 50 μL depleted serum+50 μL buffer) and incubating 30 min at RT with shaking.

Protein Assay. Protein concentrations in serum and plasma samples are measured using any protein assay procedure, e.g., the Micro BCA Method (Pierce) or Coomassie Plus (Bradford) Method (Pierce), following the manufacturer's instructions.

Serum Pre-fractionation. If desired, serum ise pre-fractionated on an anion exchange resin according to the protocol provided with the Ciphergen, Inc. EDM-Serum Fractionation Kit. Buffers needed for this protocol include: U9 Buffer (9M urea, 2% CHAPS, 50 mM Tris-HCl pH 9); Rehydration Buffer, (50 mM Tris-HCl, pH 9); Wash buffer 1 (50 mM Tris-HCl with 0.1% OGP pH 9); Wash buffer 2 (50 mM Hepes with 0.1% OGP pH 7); Wash buffer 3 (100 mM NaAcetate with 0.1% OGP pH 5); Wash buffer 4 (100 mM NaAcetate with 0.1% OGP pH 4); Wash buffer 5 (50 mM NaCitrate with 0.1% OGP pH 3); and Wash buffer 6 (33.3% isopropanol/16.7% acetonitrile/0.1% trifluoracetic acid). Wash buffer 6 should not be aliquoted for use until Wash buffer 5 has been applied to the resin to avoid evaporation of the volatile organic solvent. Materials needed for this protocol include: a 96-well filtration plate filled with dehydrated Q CERAMIC HYPERD F sorbent; microplate sealing strips; a v-bottom 96-well microplate labeled “samples”; v-bottom 96-well microplates labeled F1 through F6; 96-well microplate for collection of waste; adhesive sealing film for microplates (e.g., E&K Scientific Cat. No. T396100); a 12 column, partitioned buffer reservoir (e.g., Innovative Microplate Cat. No. S30019); pipette tips; a MicroMix 5 or equivalent mixer; a BIOMEK 2000 Laboratory Automation Workstation with PROTEINCHIP Biomarker Integration Package (optional); and a vacuum manifold (for manual use). When using a bioprocessor, make sure there are no air bubbles in the wells. To avoid introducing bubbles, the pipette tip is lowered very close to the spot surface while dispensing samples. The wells are completely emptied between washes. To ensure thorough mixing of sample with anion exchange resin, the 96 well plate was centrifuged at low speed for a few minutes.

The serum samples are thawed to ambient temperature, and then centrifuged at 20,000 g for 10 min at 4° C. 20 μL of serum sample is aliquoted to each well of a standard v-bottom 96-well microplate. 30 μL of U9 Buffer is added to each well, the microplate is covered with adhesive sealing film and mixed on the MicroMix 5 (set at 20, 5, 20) or equivalent mixer for 20 min at 4° C.

The filtration plate is tapped on the bench several times to make sure that all of the dry Q HYPERD F beads settled to the bottom of the plate. The filtration plate is taken out of the pouch and the top seal on the filtration plate is carefully removed. With an 8-channel pipette, 200 μL of Rehydration Buffer is added to each well. The filtration plate is mixed on the MicroMix 5 (set at 20, 7, 60) or equivalent mixer for 60 min at RT. The waste collection plate is placed underneath the filtration plate and a vacuum is applied to remove the buffer from the filtration plate. 200 μL of Rehydration Buffer is added to each well, and a vacuum is applied to remove the buffer in the filtration plate. This step is repeated three times, followed by washing the Q HYPERD F beads with U1 Solution (1:9 dilution of U9 Buffer in Rehydration Buffer). Q HYPERD F beads are stored in 50 mM Tris-HCl pH 9 in a 50% suspension, equilibrated by adding 125 μL Q HYPERD F beads to each well in the filter plate and then filtering the buffer, adding 150 μL U1 Solution to each well and then filtering the buffer. The U1 Solution wash is repeated three times.

50 μL of sample from each well of the sample microplate is transferred to the corresponding well in the 96-well filtration plate. 50 μL of U1 Solution is added to each well of the sample microplate, mixed 5 times, and then transferred to the corresponding well in the 96-well filtration plate. The filtration plate is covered adhesive sealing film and mixed on MicroMix 5 (set at 20, 7, 30) or equivalent mixer for 30 min at 4° C.

Fraction 1 is prepared by placing the 96-well microplate labeled F1 underneath the filtration plate, applying a vacuum and collecting the flow through into the F1 plate, adding 100 μL of Wash Buffer 1 to each well of the filtration plate, mixing for 10 min on MicroMix 5 (set at 20, 7, 10) or equivalent mixer at RT, and applying a vacuum and collecting the eluant into the F1 plate. Fraction 1 contains the flow-through and the pH 9 eluant.

Fraction 2 is prepared by adding 100 μL of Wash Buffer 2 to each well of the filtration plate, mixing for 10 min on MicroMix 5 (set at 20, 7, 10) or equivalent mixer at RT, placing the 96-well microplate labeled F2 underneath the filtration plate, and applying a vacuum and collecting the eluant into the F2 plate. This step is repeated once. Fraction 2 contains the pH 7 eluant.

Fraction 3 is prepared by adding 1000, of Wash Buffer 3 to each well of the filtration plate, mixing for 10 min on MicroMix 5 or equivalent mixer (set at 20, 7, 10) at RT, placing the 96-well microplate labeled F3 underneath the filtration plate, and applying a vacuum and collecting the eluant into the F3 plate. This step is repeated once. Fraction 3 contains the pH 5 eluant.

Fraction 4 is prepared by adding 100 μL of Wash Buffer 4 to each well of the filtration plate, mixing for 10 min on MicroMix 5 (set at 20, 7, 10) or equivalent mixer at RT, placing the 96-well microplate labeled F4 underneath the filtration plate, and applying a vacuum and collecting the eluant into the F4 plate. This step is repeated once. Fraction 4 contains the pH 4 eluant.

Fraction 5 is prepared by adding 100 μL of Wash Buffer 5 to each well of the filtration plate, mixing for 10 min on MicroMix 5 (set at 20, 7, 10) or equivalent mixer at RT, placing the 96-well microplate labeled F5 underneath the filtration plate, and applying a vacuum and collecting the eluant into the F5 plate. This step is repeated once. Fraction 5 contains the pH 3 eluant.

Fraction 6 is prepared by adding 100 μL of Wash Buffer 6 to each well of the filtration plate, mixing for 10 min on MicroMix 5 (set at 20, 7, 10) or equivalent mixer at RT, placing the 96-well microplate labeled F6 underneath the filtration plate, and applying a vacuum and collecting the eluant into the F6 plate. This step is repeated once. Fraction 6 contains the organic solvent eluant.

The six collection microplates are stored until proceeding with the PROTEINCHIP Array binding or equivalent protocol. If the samples are to be analyzed within 24 hours, store at 4° C., longer term storage should be at −20° C.

### Example 3

### Preparation of Biochips

The following protocols are used to prepare the biochips used to identify and characterize the biomarkers of the invention.

IMAC-Cu CHIP Spot Protocol. If needed, each spot of the array is outlined with a PAP wax pen and allowed to air dry. 5 μL of 100 mM copper sulfate is loaded onto each spot and incubated in a humidity chamber for 15 min. The solution is not allowed to dry. The loading process is repeated once. The loaded array is rinsed in running DW for about 10 sec. to remove excess copper. The spots are then rinsed (pipetting and aspirating) with an excess (5 to 10 μL) of 50 mM sodium acetate, pH 4 followed by aspiration. The array is rinsed in running DW for about 10 sec. 5 μL of 0.5M NaCl in PBS (binding buffer) is added to each spot, incubated for 5 min, and then excess buffer is removed by aspiration without touching the active surface. Samples are diluted 5× with 0.5 M NaCl in PBS (binding buffer) and 2 to 3 μL sample is applied per spot. The array is incubated in a humidity chamber for 30 min at RT, then the array is washed 5 times (pipetting & aspirating) with 5 μL binding buffer, followed by washing twice (pipetting & aspirating) with 5 μL DW. The array is tapped on the benchtop to remove excess water droplets, then wiped dry around the spots, taking care not to smudge the wax circles. The EAM is applied while the spots are still moist following the procedure below.

IMAC30 PROTEINCHIP Array. The IMAC30 PROTEINCHIP Array is placed into the Bioprocessor (Ciphergen, Inc., Cat. No. C503-0008-8-well, C503-0006-96-well) and 50 μL of 0.1M CuSO₄ is added to each well (volumes are adjusted depending on whether an 8 or 16 spot array is used), and incubated for 10 mM at RT with vigorous shaking (e.g., 250 rpm, or on a MicroMix, setting 20/7). Immediately after removing the copper solution from the wells, 150-250 μL, of de-ionized (DI) water is added to each well, and incubated for 2 min at RT with vigorous shaking. This step is repeated once. Immediately after removing the DI water from the wells, 150-250 μL of 0.1M sodium acetate buffer pH 4 (neutralization buffer) is added to each well, and incubated for 5 min at RT with vigorous shaking. Immediately after removing the buffer solution from the wells, 150-250 μL of DI water is added to each well, and incubated for 2 min at RT with vigorous shaking. Immediately after removing the DI water from the wells, 150-250 μL of binding buffer is added to each well, and incubated for 5 min at RT with vigorous shaking. Immediately after removing the binding buffer from the wells, 50-150 μL of sample (fractions diluted 1:5 with 0.5M NaCl in PBS binding buffer; the total protein concentration was 50-2000 μg/mL in binding buffer) is added to each well, and incubated 30 min at RT with vigorous shaking. After removing the samples from the wells, the wells are washed with 150-250 μL binding buffer for 5 min with agitation. This step is repeated twice. The wells are drained, and the array is removed from the Bioprocessor and allowed to air dry for 15-20 min. 1 μL EAM solution is applied per spot (two applications of EAM solution can be used in order to increase the peak intensity), and allowed to air dry.

CM10 PROTEINCHIP Array. Buffers needed for this protocol include: Binding buffer (100 mM sodium acetate, pH 6, 50 mM Tris-base, pH 8.5, or 50 mM Tris-HCl, pH 9.5); Sodium/ammonium acetate buffer (10-100 mM), pH 4-6; Ammonium phosphate buffer (10-100 mM), pH 6-8; HEPES Buffer, pH 7 (50 mM); Tris-HCl buffer (10-100 mM), pH 7.5-9. The CM10 PROTEINCHIP Array is placed in the Bioprocessor (Ciphergen, Inc., Cat. No. C503-0008, 8-well; C503-0006, 96-well) and 150-250 μL Binding buffer is added to each well, and incubated for 5 min at RT with vigorous shaking (e.g., 250 rpm, or on a MicroMix1, setting 20/7). This washing step is repeated once. Immediately after removing the Binding buffer, 50-150 μL sample (50-2000 μg/mL total protein, diluted in Binding buffer) is added to each well, and incubated 30 min at RT with vigorous shaking. After removing the samples from the wells, the wells were washed with 150-250 μL Binding buffer for 5 min at RT, with agitation. This step is repeated twice. After removing the Binding buffer from the wells, the wells are washed with 150-250 μL DI water for 5 min at RT, with agitation. The wells are drained, and the array is removed from the Bioprocessor and allowed to air dry for 15-20 min. 1 μL SPA solution is applied per spot. After 5 min, a second 1 μL of SPA is applied per spot, and allowed to air dry.

EAM Preparation and Application.

Saturated CHCA. A premixed solution of 100 μL ACN+100 μL 1% TFA) is added to a pre-weighed CHCA tube, vortexed for 5 min, and centrifuged for 1 min at 10,000 g. The supernatant is removed and diluted with an equal volume of ACN+1% TFA. 1 μL CHCA is added to each spot and allowed to air dry. This step can be repeated once. 1 μL of 50%, 25% or 10% saturated CHCA solution can be used to detect biomarkers of lower masses.

50% Saturated SPA. A premixed solution of 200 μL ACN+200 μL 1% TFA is added to a pre-weighed SPA tube, and vortexed for 5 min. 1 μL 50% SPA is added to each spot, and allowed to air dry. This step is repeated once.

### Example 4

### Data Collection and Analysis

The following protocols are used in preparation for collecting and analyzing the data leading to the identification and characterization of the biomarkers of the invention.

Data Collection.

1. Check laser energy and use signal enhancer. Two laser energies are used to read the arrays. A low laser energy allows peaks in the low mass range (2-20 kDa) to be well visualized, while a high laser energy improves visualization of peaks in the high mass range (>20 kDa). The signal enhancer features of PROTEINCHIP Software 3.x can be turned on to further improve visualization of higher mass species in all acquired spectra.

2. Check the appearance of spectra. The intensity and shape of the peaks should be noted. Peaks with flat tops or with non-normalized, baseline subtracted laser intensities greater than 60 generally are unreliable, since individual laser shots were probably off-scale.

3. Perform a pre-qualification run. The PROTEINCHIP Reader parameters that require the most characterization are laser energy, detector sensitivity, and detector voltage. Spot protocols including specific energy settings should be determined by performing a pre-qualification run prior to the start of the study. A pre-qualification run consists of spotting a standard sample (generally the same one used for monitoring of the project) onto a series of arrays and reading these at a range of laser energies and detector sensitivities and voltages.

Data Analysis.

1. Choose five calibrants. Mass calibration should be performed by using five calibrants in the mass range of interest. Different calibrants should be used for the low mass range versus the high mass range.

2. Normalize intensity values. Total ion current normalization should be used to normalize intensity values. Use a mass range appropriate to the analysis, but always omit the matrix region.

3. Match time lag settings. Acquire data for calibration at the various time lag focusing settings that match the actual time lag focusing settings used to read the arrays containing the samples (and the Detector voltage settings should you end up changing this between spot protocols).

4. Choose baseline subtraction setting. Use a baseline subtraction setting of eight times the fitting width.

5. Find peaks. Data analysis requires a series of preprocessing steps, including baseline subtraction, mass calibration and total ion current normalization. Once these have been performed the true data analysis can be done, i.e., finding peaks and determining their value in classifying samples. Spectra that do not show good binding of sample should be considered as unrepresentative and therefore not included in the analysis.

Sequence of Steps During Preparation Phase. Based on information collected so far, the preparation for experiments can be conducted per the following scheme.

1. Detector. Voltage is optimized using IgG QC chips. Optimization is performed periodically (e.g., weekly).

2. Mass calibration. Spot one complete NP20 A-P array with protein MW standard, using SPA for >20 KDa mass calibration, and verify spot-to-spot calibration. Spot one complete NP20 A-P array with peptide MW standard, using CHCA for <10 kDa mass calibration, and verify spot-to-spot calibration. The mass calibration is performed each time chips are analyzed on the Bioprocessor.

3. Test dilution effect. With a pooled serum sample, deplete albumin and IgG or pre-fractionate per procedures outlined above if desired, then assay protein content. Use “extraction buffer” to perform 1:1, 1:2, 1:5, 1:10 dilutions, spot samples onto various chip arrays, use CHCA and SPA with low and high laser settings to analyze spots, and choose the best dilution for each chip surface and EAM.

4. Test binding conditions I. With 2 control and 2 test serum samples, deplete albumin and IgG or pre-fractionate per procedures outlined above if desired, then assay protein content. Dilute the samples per results from step 3 above. Using selected chip arrays, choose different binding conditions, use CHCA and SPA, optimize low and high energy laser settings and sensitivity with previously determined detector voltage in step 1 above, and choose the best binding conditions for each chip surface and EAM.

5. Test binding conditions II. With 4 control and 4 test serum samples; including those analyzed in step 4 above, spot in duplicate. Deplete albumin and IgG or pre-fractionate per procedures outlined above if desired, then assay protein content. Dilute the samples per results from step 3 above. Using binding conditions for each chip surface and EAM as determined in step 4 above, optimize low and high energy laser settings and sensitivity with previously determined detector voltage, check for reproducibility (including spectra from step 4), and choose and save optimized MS settings.

### Example 5

### Serum Biomarkers for Abdominal Aortic Aneurysm (AAA)

The following protocol was used to generate mass spectra from the serum of 15 individuals, 7 of whom were diagnosed with AAA and 8 of whom were age-matched controls.

Biochip Binding Protocol. The IMAC30 PROTEINCHIP Array (Ciphergen, Inc.) was prepared as described in Example 3. Serum samples were bound to the IMAC30 Cu array basically as described in Example 3.

Energy absorbing molecules (EAM), frequently referred to as “matrix,” were added to the IMAC30 Cu array as follows. The bioprocessor's top and gasket were removed and the array was allowed to air dry. For the cyano-hydroxy-cinnamic acid (CHCA) matrix, 1 μL of 50% CHCA dissolved in 50% Acetonitrile+0.25% TFA was added to each spot in the array and allowed to air dry. 1 μL of 35% CHCA was added to each spot and allowed to air dry. For the sinapinic acid (SPA) matrix, 1 μL 50% SPA in 50% Acetonitrile and 0.5% TFA was added to each spot in the array and allowed to air dry. 1 μL 50% SPA was added to each spot and allowed to air dry.

Data Acquisition Settings. The conditions for data acquisition for the IMAC30 Cu array were determined following the protocols described in Example 4.

Identification of Biomarkers. The spectra obtained were analyzed by standard mass spectroscopy analytic methods, e.g., using the CIPHERGEN EXPRESS Data Manager Software with BIOMARKER WIZARD and Biomarker Pattern Software from Ciphergen Biosystems, Inc. The mass spectra for each group were subjected to scatter plot analysis. A Student's t-test analysis was employed to compare AAA and control groups for each protein cluster in the scatter plot, and proteins were selected that differed significantly (p<0.05 or p<0.1, as indicated) between the two groups.

Examples of the biomarkers thus discovered are presented in Table 1 below. The “Assay” column refers to the type of biochip to which the biomarkers bound, the type of EAM used, and the laser energy.

TABLE 1
Serum Biomarkers Associated with AAA

| No. | Mass | Assay | P-value | Up/Down |
|---|---|---|---|---|
| 1 | 2663 | IMAC-Cu 35% CHCA Low Laser | <0.01 | ↑ |
| 2 | 2685 | ″ | <0.05 | ↑ |
| 3 | 2726 | ″ | <0.01 | ↑ |
| 4 | 3350 | ″ | <0.05 | ↓ |
| 5 | 4094 | ″ | <0.01 | ↓ |
| 6 | 4646 | ″ | <0.01 | ↓ |
| 7 | 4708 | ″ | <0.05 | ↓ |
| 8 | 5131 | ″ | <0.01 | ↓ |
| 9 | 5990 | ″ | <0.01 | ↓ |
| 10 | 11573 | ″ | <0.05 | ↑ |
| 11 | 14069 | ″ | <0.01 | ↓ |
| 12 | 11445 | IMAC-Cu SPA Low Laser | <0.01 | ↑ |
| 13 | 11643 | ″ | <0.05 | ↑ |
| 14 | 11841 | ″ | <0.01 | ↑ |
| 15 | 12506 | ″ | <0.01 | ↓ |
| 16 | 13933 | ″ | <0.01 | ↓ |
| 17 | 14564 | ″ | <0.05 | ↑ |
| 18 | 27855 | ″ | <0.01 | ↓ |
| 19 | 55985 | ″ | <0.01 | ↓ |
| 20 | 73027 | ″ | <0.01 | ↑ |
| 21 | 94635 | ″ | <0.01 | ↓ |
| 22 | 11487 | IMAC-Cu SPA High Laser | <0.01 | ↑ |
| 23 | 11687 | ″ | <0.05 | ↑ |
| 24 | 12545 | ″ | <0.05 | ↓ |
| 25 | 13288 | ″ | <0.01 | ↑ |
| 26 | 14013 | ″ | <0.01 | ↓ |
| 27 | 14608 | ″ | <0.05 | ↑ |
| 28 | 53715 | ″ | <0.05 | ↑ |

### Example 6

### Serum and Plasma Biomarkers for Abdominal Aortic Aneurysm (AAA)

The following protocol was used to generate mass spectra from the serum and plasma of 14 individuals, 7 of whom were diagnosed with AAA and 7 of whom were age-matched controls.

Depletion of Albumin and IgG. Pre-fractionation to deplete albumin and IgG from the serum and plasma was performed as described in Example 2.

Biochip Binding Protocol. The IMAC30 and CM10 PROTEINCHIP Arrays (Ciphergen, Inc.) were prepared as described in Example 3. Serum and plasma samples were bound to IMAC30 and CM10 arrays basically as described in Example 3. The pH4 fraction for the CM10 array was prepared as described in Example 2.

EAM or matrix (SPA) were added to the IMAC30 Cu array as described in Example 5. EAM or matrix were added to the CM10 array as follows. The bioprocessor's top and gasket were removed and the array was allowed to air dry. For the SPA matrix, 400 μL of 50% acetonitrile, 0.5% TFA were added to a SPA tube and mixed for 5 min at RT. 1 μL of the mixture was added to each spot in the array and allowed to air dry. This step was repeated once.

Data Acquisition Settings. The conditions for data acquisition for the IMAC30 Cu array and the CM10 array were determined following the protocols described in Example 4.

Identification of Biomarkers. The spectra obtained were analyzed as described in Example 5. A Student's t-test analysis was employed to compare AAA and control groups for each protein cluster in the scatter plot, and proteins were selected that differed significantly (p<0.05 or p<0.1 as indicated) between the two groups.

Examples of albumin and IgG depleted biomarkers thus discovered are presented in Tables 2 (serum) and 3 (plasma) below. The “Assay” column refers to the type of biochip to which the biomarkers bound, the type of EAM used with the biochip, and the laser energy used to ionize the biomarkers.

TABLE 2
Albumin and IgG Depleted Serum Biomarkers Associated with AAA

| No. | Mass | Assay | P-value | Up/Down |
|---|---|---|---|---|
| 1 | 3129 | IMAC-Cu SPA Low Laser | <0.1 | ↓ |
| 2 | 3195 | ″ | <0.05 | ↑ |
| 3 | 3402 | ″ | <0.1 | ↑ |
| 4 | 3504 | ″ | <0.05 | ↓ |
| 5 | 3642 | ″ | <0.05 | ↓ |
| 6 | 3660 | ″ | <0.1 | ↑ |
| 7 | 3881 | ″ | <0.05 | ↓ |
| 8 | 4450 | ″ | <0.1 | ↓ |
| 9 | 5753 | ″ | <0.1 | ↓ |
| 10 | 8858 | ″ | <0.1 | ↑ |
| 11 | 11638 | ″ | <0.1 | ↑ |
| 12 | 108804 | ″ | <0.05 | ↓ |
| 13 | 14631 | IMAC-Cu SPA High Laser | <0.1 | ↑ |
| 14 | 15480 | ″ | <0.05 | ↓ |
| 15 | 155279 | ″ | <0.1 | ↑ |
| 16 | 3003 | CM10 pH 4 SPA Low Laser | <0.05 | ↑ |
| 17 | 3061 | ″ | <0.05 | ↑ |
| 18 | 3233 | ″ | <0.05 | ↓ |
| 19 | 3685 | ″ | <0.05 | ↑ |
| 20 | 4144 | ″ | <0.1 | ↑ |
| 21 | 4429 | ″ | <0.1 | ↑ |
| 22 | 4490 | ″ | <0.05 | ↑ |
| 23 | 4558 | ″ | <0.1 | ↑ |
| 24 | 4603 | ″ | <0.05 | ↓ |
| 25 | 7498 | ″ | <0.1 | ↓ |
| 26 | 7698 | ″ | <0.05 | ↓ |
| 27 | 8076 | ″ | <0.1 | ↓ |
| 28 | 9211 | ″ | <0.05 | ↓ |
| 29 | 13713 | CM10 pH 4 SPA High Laser | <0.1 | ↓ |
| 30 | 13819 | ″ | <0.1 | ↓ |
| 31 | 15720 | ″ | <0.1 | ↓ |
| 32 | 39702 | ″ | <0.05 | ↑ |

TABLE 3
Albumin and IgG Depleted Plasma Biomarkers Associated with AAA

| No. | Mass | Assay | P-value | Up/Down |
|---|---|---|---|---|
| 33 | 3072 | IMAC-Cu SPA Low Laser | <0.1 | ↑ |
| 34 | 3211 | ″ | <0.1 | ↓ |
| 35 | 3247 | ″ | <0.05 | ↓ |
| 36 | 3285 | ″ | <0.05 | ↓ |
| 37 | 3307 | ″ | <0.1 | ↓ |
| 38 | 3443 | ″ | <0.05 | ↓ |
| 39 | 3508 | ″ | <0.05 | ↓ |
| 40 | 3658 | ″ | <0.1 | ↓ |
| 41 | 3701 | ″ | <0.1 | ↓ |
| 42 | 3748 | ″ | <0.1 | ↑ |
| 43 | 3935 | ″ | <0.1 | ↓ |
| 44 | 4030 | ″ | <0.05 | ↑ |
| 45 | 4632 | ″ | <0.05 | ↓ |
| 46 | 4703 | ″ | <0.1 | ↓ |
| 47 | 4750 | ″ | <0.05 | ↓ |
| 48 | 11635 | ″ | <0.05 | ↑ |
| 49 | 14579 | ″ | <0.05 | ↑ |
| 50 | 66262 | IMAC-Cu SPA High Laser | <0.05 | ↑ |
| 51 | 3062 | CM10 pH 4 SPA Low Laser | <0.1 | ↑ |
| 52 | 3088 | ″ | <0.1 | ↑ |
| 53 | 3092 | ″ | <0.05 | ↑ |
| 54 | 3204 | ″ | <0.1 | ↑ |
| 55 | 3253 | ″ | <0.1 | ↓ |
| 56 | 3284 | ″ | <0.05 | ↓ |
| 57 | 3303 | ″ | <0.05 | ↓ |
| 58 | 3458 | ″ | <0.05 | ↓ |
| 59 | 3600 | ″ | <0.1 | ↑ |
| 60 | 3681 | ″ | <0.05 | ↑ |
| 61 | 3708 | ″ | <0.1 | ↑ |
| 62 | 3867 | ″ | <0.05 | ↑ |
| 63 | 3943 | ″ | <0.05 | ↓ |
| 64 | 4493 | ″ | <0.05 | ↑ |
| 65 | 4602 | ″ | <0.05 | ↓ |
| 66 | 4686 | ″ | <0.1 | ↑ |
| 67 | 6072 | ″ | <0.05 | ↑ |
| 68 | 6412 | ″ | <0.05 | ↑ |
| 69 | 6559 | ″ | <0.05 | ↑ |
| 70 | 6608 | ″ | <0.05 | ↑ |
| 71 | 9632 | ″ | <0.1 | ↑ |
| 72 | 41006 | ″ | <0.1 | ↑ |
| 73 | 72882 | ″ | <0.1 | ↑ |
| 74 | 10791 | CM10 pH 4 SPA High Laser | <0.05 | ↑ |
| 75 | 11631 | ″ | <0.05 | ↑ |
| 76 | 11677 | ″ | <0.05 | ↑ |
| 77 | 14626 | ″ | <0.05 | ↑ |
| 78 | 37177 | ″ | <0.1 | ↑ |
| 79 | 51037 | ″ | <0.1 | ↑ |

### Example 7

### Urine Biomarkers for Abdominal Aortic Aneurysm (AAA)

The following protocol was used to generate mass spectra from the urine of 14 individuals, 7 of whom were diagnosed with AAA and 7 of whom were age-matched controls.

Biochip Binding Protocol. The IMAC30 and CM10 PROTEINCHIP Arrays (Ciphergen, Inc.) were prepared as described in Example 3. Urine samples were bound to IMAC30 Cu and CM10 arrays basically as described in Example 3. The pH 4 fraction for the CM10 array was prepared as described in Example 2.

EAM or matrix (CHCA) were added to the IMAC30 Cu array as described in Example 5, except the concentrations of CHCA were 35% and 15%. EAM or matrix (SPA) were added to the CM10 array as described in Example 6.

Data Acquisition Settings. The conditions for data acquisition for the IMAC30 Cu array and the CM10 array were determined following the protocols described in Example 4.

Identification of Biomarkers. The spectra obtained were analyzed as described in Example 5. A Student's t-test analysis was employed to compare AAA and control groups for each protein cluster in the scatter plot, and proteins were selected that differed significantly (p<0.05 or p<0.01, as indicated) between the two groups.

Examples of the biomarkers thus discovered are presented in Table 4 below. The “Assay” column refers to the type of biochip to which the biomarkers bound, the type of EAM used with the biochip, and the laser energy used to ionize the biomarkers.

TABLE 4
Urine Biomarkers Associated with AAA

| No. | Mass | Assay | P-value | Up/Down |
|---|---|---|---|---|
| 1 | 3360 | CM10 pH 4 SPA Low Laser | <0.1 | ↑ |
| 2 | 3742 | ″ | <0.05 | ↑ |
| 3 | 3856 | ″ | <0.05 | ↑ |
| 4 | 4381 | ″ | <0.1 | ↑ |
| 5 | 4458 | ″ | <0.05 | ↓ |
| 6 | 4734 | ″ | <0.1 | ↓ |
| 7 | 5396 | ″ | <0.1 | ↑ |
| 8 | 5515 | ″ | <0.1 | ↑ |
| 9 | 5704 | ″ | <0.1 | ↑ |
| 10 | 5716 | ″ | <0.05 | ↑ |
| 11 | 6033 | ″ | <0.1 | ↓ |
| 12 | 6202 | ″ | <0.1 | ↓ |
| 13 | 13391 | ″ | <0.1 | ↓ |
| 14 | 45878 | ″ | <0.1 | ↑ |
| 15 | 91571 | ″ | <0.1 | ↑ |
| 16 | 1293 | IMAC-Cu 35% CHCA | <0.1 | ↑ |
| 17 | 1914 | ″ | <0.1 | ↓ |
| 18 | 2407 | ″ | <0.1 | ↑ |
| 19 | 2746 | ″ | <0.05 | ↑ |
| 20 | 4700 | ″ | <0.1 | ↓ |
| 21 | 5624 | ″ | <0.1 | ↑ |
| 22 | 9625 | ″ | <0.1 | ↑ |
| 23 | 179 | IMAC-Cu 15% CHCA | <0.1 | ↑ |
| 24 | 220 | ″ | <0.05 | ↑ |
| 25 | 423 | ″ | <0.05 | ↓ |
| 26 | 445 | ″ | <0.1 | ↓ |
| 27 | 1027 | ″ | <0.1 | ↑ |

### Example 8

### Biomarkers Associated with Abdominal Aortic Aneurysm (AAA) and Age-Related Macular Degeneration (AMD)

Age-related macular degeneration (AMD), which is a degenerative condition of a specialized region of the central retina called the macula, is the leading cause of blindness in adults over 60, affecting more than 50 million people worldwide (Klein et al., Am J Ophthalmol. 137:486, 2004). Early AMD is characterized by the thinning of the macula and formation of deposits called drusen in the macula. Most people with early AMD have good vision. Persons with drusen may develop advanced AMD, which is associated with profound vision loss. Advanced AMD has two forms: dry, which is a slow, degenerative process with gradual central vision loss due to loss of photoreceptors; and wet, which is associated with sudden vision loss due to abnormal blood vessel growth (i.e., choroidal neovascularization) under the macula.

Some individuals that have been diagnosed with AAA also have been diagnosed with AMD. As described below, it has been found that several biomarkers are present at different levels in the serum, plasma or urine of individuals diagnosed with both AAA and AMD, compared to age-matched controls. Such biomarkers may be useful to diagnose individuals as having both AAA and AMD. This information may be useful in designing treatment strategies for AAA/AMD patients.

The following protocol was used to generate mass spectra from: (A) the serum of 15 individuals, 7 of whom were diagnosed with both AAA and AMD and 8 of whom were age-matched controls (Table 5, Nos. 1-32); (B) the urine of 14 individuals, 7 of whom were diagnosed with both AAA and AMD and 7 of whom were age-matched controls (Table 5, Nos. 33-58); and (C) the serum and plasma of 14 individuals, 7 of whom were diagnosed with both AAA and AMD and 7 of whom were age-matched controls (Table 5, Nos. 59-192).

Depletion of Albumin and IgG. Pre-fractionation to deplete albumin and IgG from the serum and plasma in (C) was performed as described in Example 6.

Biochip Binding Protocol. The IMAC30 and CM10 PROTEINCHIP Arrays (Ciphergen, Inc.) were prepared as described in Example 3. Serum and plasma samples were bound to the IMAC30 Cu and CM10 arrays basically as described in Example 3. The pH 4 fraction for the CM10 array was prepared as described in Example 2.

EAM or matrix (CHCA or SPA) were added to the IMAC30 Cu array and to the CM10 array as described in Example 5. The final CHCA concentration was 35% or 15%, as indicated in Table 5.

Data Acquisition Settings. The conditions for data acquisition for the IMAC30 Cu array and the CM10 array were determined following the protocols described in Example 4.

Identification of Biomarkers. The spectra obtained were analyzed as described in Example 5. A Student's t-test analysis was employed to compare AMD/AAA and control groups for each protein cluster in the scatter plot, and proteins were selected that differed significantly (p<0.05 or p<0.1, as indicated) between the two groups.

Examples of the biomarkers thus discovered are presented in Table 5 below. The “Source” column refers to source of the biomarkers, i.e., serum, plasma or urine. The “Assay” column refers to the type of biochip to which the biomarkers bound, the type of EAM used with the biochip, and the laser energy used to ionize the biomarkers.

TABLE 5
Biomarkers Associated with Both AAA and AMD

| No. | Mass | Source | Assay | P-value | Up/Down |
|---|---|---|---|---|---|
| 1 | 2935 | Serum | IMAC-Cu 35% CHCA Low Laser | <0.05 | ↓ |
| 2 | 3318 | | ″ | <0.1 | ↓ |
| 3 | 3329 | | ″ | <0.05 | ↓ |
| 4 | 3350 | | ″ | <0.05 | ↓ |
| 5 | 3949 | | ″ | <0.1 | ↓ |
| 6 | 3958 | | ″ | <0.05 | ↓ |
| 7 | 4094 | | ″ | <0.1 | ↓ |
| 8 | 4284 | | ″ | <0.05 | ↓ |
| 9 | 4347 | | ″ | <0.05 | ↓ |
| 10 | 4646 | | ″ | <0.05 | ↓ |
| 11 | 6190 | | ″ | <0.05 | ↑ |
| 12 | 6652 | | ″ | <0.1 | ↑ |
| 13 | 11753 | | ″ | <0.1 | ↑ |
| 14 | 6233 | Serum | IMAC-Cu SPA Low Laser | <0.05 | ↑ |
| 15 | 6470 | | ″ | <0.1 | ↓ |
| 16 | 8739 | | ″ | <0.05 | ↓ |
| 17 | 9593 | | ″ | <0.05 | ↓ |
| 18 | 11010 | | ″ | <0.1 | ↓ |
| 19 | 11643 | | ″ | <0.05 | ↑ |
| 20 | 11841 | | ″ | <0.05 | ↑ |
| 21 | 42832 | | ″ | <0.1 | ↓ |
| 22 | 118128 | | ″ | <0.1 | ↓ |
| 23 | 11487 | Serum | IMAC-Cu SPA High Laser | <0.1 | ↑ |
| 24 | 12545 | | ″ | <0.05 | ↓ |
| 25 | 13681 | | ″ | <0.05 | ↓ |
| 26 | 13812 | | ″ | <0.05 | ↓ |
| 27 | 14013 | | ″ | <0.05 | ↓ |
| 28 | 18252 | | ″ | <0.05 | ↑ |
| 29 | 22682 | | ″ | <0.1 | ↓ |
| 30 | 35905 | | ″ | <0.05 | ↑ |
| 31 | 39572 | | ″ | <0.1 | ↑ |
| 32 | 146602 | | ″ | <0.1 | ↑ |
| 33 | 2672 | Urine | CM10 pH 4 SPA Low Laser | <0.1 | ↑ |
| 34 | 4062 | | ″ | <0.05 | ↑ |
| 35 | 4311 | | ″ | <0.1 | ↓ |
| 36 | 4458 | | ″ | <0.1 | ↓ |
| 37 | 5704 | | ″ | <0.1 | ↑ |
| 38 | 5742 | | ″ | <0.05 | ↑ |
| 39 | 6253 | | ″ | <0.05 | ↓ |
| 40 | 45878 | | ″ | <0.1 | ↑ |
| 41 | 60948 | | ″ | <0.1 | ↓ |
| 42 | 91571 | | ″ | <0.1 | ↑ |
| 43 | 1914 | Urine | IMAC-Cu 35% CHCA Low Laser | <0.1 | ↓ |
| 44 | 3027 | | ″ | <0.1 | ↓ |
| 45 | 4602 | | ″ | <0.1 | ↑ |
| 46 | 5916 | | ″ | <0.1 | ↓ |
| 47 | 6130 | | ″ | <0.05 | ↓ |
| 48 | 6189 | | ″ | <0.05 | ↓ |
| 49 | 9625 | | ″ | <0.1 | ↑ |
| 50 | 137 | Urine | IMAC-Cu 15% CHCA Low Laser | <0.1 | ↓ |
| 51 | 179 | | ″ | <0.05 | ↓ |
| 52 | 423 | | ″ | <0.1 | ↓ |
| 53 | 430 | | ″ | <0.1 | ↑ |
| 54 | 445 | | ″ | <0.1 | ↓ |
| 55 | 461 | | ″ | <0.05 | ↑ |
| 56 | 637 | | ″ | <0.1 | ↑ |
| 57 | 643 | | ″ | <0.1 | ↑ |
| 58 | 671 | | ″ | <0.05 | ↓ |
| 59 | 3035 | Serum* | IMAC-Cu SPA Low Laser | <0.1 | ↓ |
| 60 | 3314 | | ″ | <0.1 | ↑ |
| 61 | 3941 | | ″ | <0.1 | ↓ |
| 62 | 4100 | | ″ | <0.05 | ↑ |
| 63 | 4346 | | ″ | <0.1 | ↓ |
| 64 | 4450 | | ″ | <0.05 | ↓ |
| 65 | 5290 | | ″ | <0.1 | ↓ |
| 66 | 5814 | | ″ | <0.05 | ↓ |
| 67 | 5836 | | ″ | <0.05 | ↑ |
| 68 | 6378 | | ″ | <0.1 | ↓ |
| 69 | 6391 | | ″ | <0.1 | ↓ |
| 70 | 6557 | | ″ | <0.1 | ↓ |
| 71 | 7501 | | ″ | <0.05 | ↓ |
| 72 | 7857 | | ″ | <0.1 | ↓ |
| 73 | 7905 | | ″ | <0.1 | ↓ |
| 74 | 8858 | | ″ | <0.1 | ↑ |
| 75 | 9211 | | ″ | <0.05 | ↓ |
| 76 | 9414 | | ″ | <0.1 | ↓ |
| 77 | 11638 | | ″ | <0.05 | ↑ |
| 78 | 11834 | | ″ | <0.05 | ↑ |
| 79 | 125870 | | ″ | <0.1 | ↑ |
| 80 | 12557 | Serum* | IMAC-Cu SPA High Laser | <0.05 | ↓ |
| 81 | 13837 | | ″ | <0.1 | ↓ |
| 82 | 15480 | | ″ | <0.05 | ↓ |
| 83 | 23562 | | ″ | <0.1 | ↑ |
| 84 | 34270 | | ″ | <0.1 | ↑ |
| 85 | 36008 | | ″ | <0.1 | ↑ |
| 86 | 37788 | | ″ | <0.1 | ↑ |
| 87 | 44593 | | ″ | <0.1 | ↑ |
| 88 | 3061 | Serum* | CM10 pH 4 SPA Low Laser | <0.1 | ↑ |
| 89 | 3189 | | ″ | <0.1 | ↓ |
| 90 | 3507 | | ″ | <0.1 | ↓ |
| 91 | 3685 | | ″ | <0.1 | ↑ |
| 92 | 3849 | | ″ | <0.05 | ↓ |
| 93 | 4132 | | ″ | <0.1 | ↓ |
| 94 | 4144 | | ″ | <0.05 | ↑ |
| 95 | 4490 | | ″ | <0.1 | ↑ |
| 96 | 4603 | | ″ | <0.05 | ↓ |
| 97 | 4775 | | ″ | <0.1 | ↑ |
| 98 | 5873 | | ″ | <0.1 | ↓ |
| 99 | 6377 | | ″ | <0.1 | ↓ |
| 100 | 6778 | | ″ | <0.05 | ↓ |
| 101 | 6823 | | ″ | <0.05 | ↓ |
| 102 | 7498 | | ″ | <0.1 | ↓ |
| 103 | 7698 | | ″ | <0.05 | ↓ |
| 104 | 8076 | | ″ | <0.1 | ↓ |
| 105 | 8693 | | ″ | <0.05 | ↓ |
| 106 | 9211 | | ″ | <0.05 | ↓ |
| 107 | 9414 | | ″ | <0.1 | ↓ |
| 108 | 13771 | | ″ | <0.05 | ↓ |
| 109 | 25454 | | ″ | <0.1 | ↑ |
| 110 | 10508 | Serum* | CM10 pH 4 SPA High Laser | <0.1 | ↓ |
| 111 | 13713 | | ″ | <0.1 | ↓ |
| 112 | 18291 | | ″ | <0.1 | ↑ |
| 113 | 39702 | | ″ | <0.05 | ↑ |
| 114 | 40545 | | ″ | <0.1 | ↑ |
| 115 | 73047 | | ″ | <0.1 | ↓ |
| 116 | 3123 | Plasma* | IMAC-Cu SPA Low Laser | <0.05 | ↑ |
| 117 | 3188 | | ″ | <0.05 | ↓ |
| 118 | 3247 | | ″ | 0.1 | ↓ |
| 119 | 3285 | | ″ | <0.05 | ↓ |
| 120 | 3307 | | ″ | 0.1 | ↓ |
| 121 | 3387 | | ″ | <0.05 | ↓ |
| 122 | 3474 | | ″ | <0.05 | ↑ |
| 123 | 3658 | | ″ | <0.05 | ↓ |
| 124 | 3748 | | ″ | <0.05 | ↑ |
| 125 | 3822 | | ″ | 0.1 | ↓ |
| 126 | 3850 | | ″ | 0.1 | ↓ |
| 127 | 3935 | | ″ | 0.1 | ↓ |
| 128 | 4133 | | ″ | 0.1 | ↑ |
| 129 | 4632 | | ″ | 0.1 | ↓ |
| 130 | 5447 | | ″ | <0.05 | ↑ |
| 131 | 5789 | | ″ | 0.1 | ↑ |
| 132 | 5856 | | ″ | 0.1 | ↑ |
| 133 | 5916 | | ″ | <0.05 | ↑ |
| 134 | 7501 | | ″ | 0.1 | ↓ |
| 135 | 7698 | | ″″ | <0.05 | ↓ |
| 136 | 7768 | | ″ | <0.05 | ↓ |
| 137 | 7903 | | ″ | <0.05 | ↓ |
| 138 | 8074 | | ″ | <0.05 | ↓ |
| 139 | 9209 | | ″ | <0.05 | ↓ |
| 140 | 9419 | | ″ | <0.05 | ↓ |
| 141 | 11635 | | ″ | <0.05 | ↑ |
| 142 | 11834 | | ″ | <0.05 | ↑ |
| 143 | 14579 | | ″ | <0.05 | ↑ |
| 144 | 13834 | Plasma* | IMAC-Cu SPA High Laser | <0.1 | ↓ |
| 145 | 15274 | | ″ | <0.05 | ↓ |
| 146 | 18295 | | ″ | <0.05 | ↑ |
| 147 | 74846 | | ″ | 0.1 | ↓ |
| 148 | 118670 | | ″ | <0.05 | ↑ |
| 149 | 3052 | Plasma* | CM10 pH 4 SPA Low Laser | <0.05 | ↓ |
| 150 | 3081 | | ″ | <0.05 | ↓ |
| 151 | 3088 | | ″ | 0.1 | ↑ |
| 152 | 3105 | | ″ | 0.1 | ↑ |
| 153 | 3113 | | ″ | <0.05 | ↑ |
| 154 | 3257 | | ″ | <0.05 | ↑ |
| 155 | 3284 | | ″ | <0.05 | ↓ |
| 156 | 3303 | | ″ | <0.05 | ↓ |
| 157 | 3317 | | ″ | <0.05 | ↓ |
| 158 | 3325 | | ″ | <0.05 | ↓ |
| 159 | 3412 | | ″ | <0.05 | ↓ |
| 160 | 3435 | | ″ | <0.05 | ↓ |
| 161 | 3475 | | ″ | 0.1 | ↓ |
| 162 | 3514 | | ″ | 0.1 | ↑ |
| 163 | 3614 | | ″ | <0.05 | ↓ |
| 164 | 3681 | | ″ | <0.05 | ↑ |
| 165 | 3709 | | ″ | <0.05 | ↓ |
| 166 | 3860 | | ″ | 0.1 | ↑ |
| 167 | 4100 | | ″ | <0.05 | ↑ |
| 168 | 4170 | | ″ | 0.1 | ↑ |
| 169 | 4187 | | ″ | 0.1 | ↓ |
| 170 | 4307 | | ″ | <0.05 | ↑ |
| 171 | 4438 | | ″ | <0.05 | ↓ |
| 172 | 4493 | | ″ | <0.05 | ↑ |
| 173 | 5059 | | ″ | 0.1 | ↑ |
| 174 | 5105 | | ″ | <0.05 | ↑ |
| 175 | 5211 | | ″ | <0.05 | ↑ |
| 176 | 6072 | | ″ | 0.1 | ↑ |
| 177 | 6559 | | ″ | <0.05 | ↑ |
| 178 | 8693 | | ″ | <0.05 | ↓ |
| 179 | 9632 | | ″ | <0.05 | ↑ |
| 180 | 11619 | | ″ | <0.05 | ↑ |
| 181 | 22087 | | ″ | 0.1 | ↑ |
| 182 | 33117 | | ″ | <0.05 | ↑ |
| 183 | 44269 | | ″ | <0.05 | ↑ |
| 184 | 145931 | | ″ | 0.1 | ↓ |
| 185 | 10791 | Plasma* | CM10 pH 4 SPA High Laser | <0.05 | ↑ |
| 186 | 11476 | | ″ | <0.05 | ↑ |
| 187 | 11631 | | ″ | 0.1 | ↑ |
| 188 | 11677 | | ″ | <0.05 | ↑ |
| 189 | 11876 | | ″ | <0.05 | ↑ |
| 190 | 14626 | | ″ | 0.1 | ↑ |
| 191 | 24295 | | ″ | 0.1 | ↑ |
| 192 | 28851 | | ″ | 0.1 | ↑ |

*Albumin and IgG depleted serum or plasma.

Although the present invention has been described in detail with reference to specific embodiments, those of skill in the art will recognize that modifications and improvements are within the scope and spirit of the invention, as set forth in the claims which follow. All publications and patent documents cited herein are incorporated herein by reference as if each such publication or document was specifically and individually indicated to be incorporated herein by reference. Citation of publications and patent documents (patents, published patent applications, and unpublished patent applications) is not intended as an admission that any such document is pertinent prior art, nor does it constitute any admission as to the contents or date of the same. The invention having now been described by way of written description, those of skill in the art will recognize that the invention can be practiced in a variety of embodiments and that the foregoing description is for purposes of illustration and not limitation of the following claims.

What is claimed is:

1. A method for diagnosing abdominal aortic aneurysm (AAA) in an individual, the method comprising:
a) determining levels of at least two AAA-associated protein markers (biomarkers) in a biological sample from the individual; and
b) comparing the levels of the at least two biomarkers to reference levels of the at least two biomarkers characteristic of a control population of individuals without AAA,
wherein a difference in the levels of the at least two biomarkers between the biological sample from the individual and the control population indicates that the individual has an increased likelihood of having AAA, and
wherein the at least two biomarkers are biomarkers listed in Table 1, 2, 3 or 4.

2. The method of claim 1, wherein the levels of the at least two biomarkers are measured by surface enhanced laser desorption ionization (SELDI).

3. The method of claim 1, wherein the biological sample is blood, serum, plasma, or urine from the individual.

4. The method of claim 3, wherein the biological sample is serum.

5. The method of claim 4, wherein the at least two biomarkers are selected from the group listed in Table 1 consisting of 2685, 3350, 4708, 11573, 11643, 14564, 11687, 12545, 14608, and 53715.

6. The method of claim 4, wherein the at least two biomarkers are selected from the group listed in Table 1 consisting of 2685, 11573, 11643, 14564, 11687, 14608, and 53715.

7. The method of claim 4, wherein the at least two biomarkers are selected from the group listed in Table 1 consisting of 3350, 4708, and 12545.

8. The method of claim 4, wherein the biological sample is serum depleted of albumin and IgG.

9. The method of claim 8, wherein the at least two biomarkers are selected from the group listed in Table 2 consisting of 3195, 3504, 3642, 3881, 108804, 15480, 3003, 3061, 3233, 3685, 4490, 4603, 7698, 9211, and 39702.

10. The method of claim 8, wherein the at least two biomarkers are selected from the group listed in Table 2 consisting of 3195, 3003, 3061, 3685, 4490, and 39702.

11. The method of claim 8, wherein the at least two biomarkers are selected from the group listed in Table 2 consisting of 3504, 3642, 3881, 108804, 15480, 3233, 4603, 7698, and 9211.

12. The method of claim 3, wherein the biological sample is plasma.

13. The method of claim 12, wherein the biological sample is plasma depleted of albumin and IgG.

14. The method of claim 13, wherein the at least two biomarkers are selected from the group listed in Table 3 consisting of 3247, 3285, 3443, 3508, 4030, 4632, 4750, 11635, 14579, 66262, 3092, 3284, 3303, 3458, 3681, 3867, 3943, 4493, 4602, 6072, 6412, 6559, 6608, 10791, 11631, 11677, and 14626.

15. The method of claim 13, wherein the at least two biomarkers are selected from the group listed in Table 3 consisting of 4030, 11635, 14579, 66262, 3092, 3681, 3867, 4493, 6072, 6412, 6559, 6608, 10791, 11631, 11677, and 14626.

16. The method of claim 13, wherein the at least two biomarkers are selected from the group listed in Table 3 consisting of 3247, 3285, 3443, 3508, 4632, 4750, 3284, 3303, 3458, 3943, and 4602.

17. The method of claim 3, wherein the biological sample is urine.

18. The method of claim 17, wherein the at least two biomarkers are selected from the group listed in Table 4 consisting of 3742, 3856, 4458, 5716, 2746, 220, and 423.

19. The method of claim 17, wherein the at least two biomarkers are selected from the group listed in Table 4 consisting of 3742, 3856, 5716, 2746, and 220.

20. The method of claim 17, wherein the at least two biomarkers listed in Table 4 are 4458 and 423.

21. The method of claim 1, wherein the at least two biomarkers are a set of biomarkers comprising at least 2, at least 3, at least 4, or at least 5 of the biomarkers listed in Table 1, 2, 3 or 4.

22. The method of claim 21, wherein the set of biomarkers comprises at least 2, at least 3, at least 4, or at least 5 of the biomarkers present at elevated levels in individuals diagnosed with AAA as compared to a control population in Table 1, 2, 3 or 4.

23. The method of claim 21, wherein the set of biomarkers comprises at least 2, at least 3, at least 4, or at least 5 of the biomarkers present at reduced levels in individuals diagnosed with AAA as compared to a control population in Table 1, 2, 3 or 4.

24. A method for monitoring the progression of AAA in an individual, comprising:
a) determining levels of at least two biomarkers listed in Tables 1-4 in a biological sample from the individual;
b) comparing the levels of the at least two biomarkers in a) to reference levels of the at least two biomarkers characteristic of a control population of individuals without AAA;
c) determining levels of the at least two biomarkers at a later time point; and
d) comparing the levels of the at least two biomarkers in c) to the reference levels of the at least two biomarkers characteristic of a control population of individuals without AAA,
wherein an increased difference in the levels of the at least two biomarkers measured in d) compared to b) indicates progression of AAA, and
wherein a decreased difference in the levels of the at least two biomarkers measured in d) compared to b) indicates regression of AAA.
